(19) United States
(12) Patent Application Publication
(10) Pub. No.: US 2006/0040924 A1
(43) Pub. Date: Feb. 23, 2006

(54) Derivatives of aryl (or heteroaryl) azolylcarbinols for the treatment of renal colic

(75) Inventor: Montserrat Abadias, Barcelona (ES)

Correspondence Address: BANNER & WITCOFF, 1001 G STREET N W, SUITE 1100, WASHINGTON, DC, 20001, US

(73) Assignee: Laboratorios Dr. Esteve S.A., Barcelona (ES)

(21) Appl. No.: 11/158,295

(22) Filed: Jun. 22, 2005

Related U.S. Application Data
(60) Provisional application No. 60/581,371, filed on Jun. 22, 2004.

(51) Int. Cl. A61K 31/5513; A61K 31/454; A61K 31/4164

(52) U.S. Cl. 514/221; 514/396; 514/326

(57) ABSTRACT

The present invention refers to the use of derivatives of aryl (or heteroaryl) azolylcarbinols of general formula (I), and their physiologically acceptable salts, as medicinal products for human and/or animal therapeutics for the treatment of renal colic.

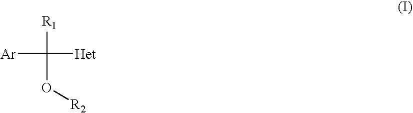

### FIELD OF THE INVENTION

The present invention refers to the use of derivatives of aryl (or heteroaryl) azolylcarbinols of general formula (I), and their physiologically acceptable salts, as medicinal products for human and/or animal therapeutics for the treatment of renal colic.

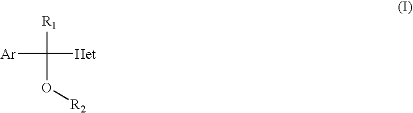

### BACKGROUND OF THE INVENTION

Renal colic is one of the most common and excruolating pains, frequently caused by an acute partial ureteral obstruction due to a calculus. Pressure of the urine tends to increase causing an increase in tension of the walls of the ureter and renal pelvis, which elicits pain. The urinary tract obstruction is a potent stimulus for renal prostaglandin synthesis, resulting in increased renal blood flow and diuresis. The emergency management of this condition aims to control the severe pain until the obstruction is relieved.

Non-steroidal anti-inflammatory drugs (NSAIDs) have been extensively evaluated to treat this condition, particularly in Europe. Most of the studies have shown comparable or better efficacy in comparison with oploids as well as an advantageous safety profile 1. However, some controversy remains at this point as prostaglandin synthetase inhibitors decrease diuresis by decreasing renal blood flow and glomerular filtration rate because of an increase in the pre-glomerular resistance. Besides, this might not be detected clinically because contralateral kidney function is not affected since NSAIDs would act selectively on the obstructed kidney and the serum creatinine may remain within normal limits.

Therefore, there is still a need for an effective treatment of renal cholic especially those with a neutral or beneficial effect on the kidney.

In our patents EP 289380 B1 (U.S. Pat. No. 6,017,698) and WO 99/52525 (U.S. Pat. No. 6,410,582) we have described derivatives of carbinols of general formula (I) with analgesic activity, whereas e.g. US 2003/022925 claims their use for urinary incontinence

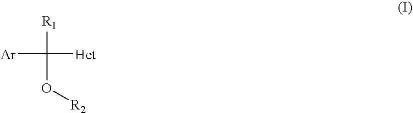

In EP 289380 B1 (U.S. Pat. No. 5,017,596) and WO 99152525 (U.S. Pat. No. 6,410.582) these compounds of general formula (I). Ar represents a benzene ring or a thiophene ring with or without substitutions, R₁ represents a hydrogen atom or a lower alkyl group from C₁ to C₄; R₂ represents a dialkylaminoalkyl or azaheterocyclylalkyl and Het represents an azole with or without substitutions, and their physiologically acceptable salts.

In our patent applications WO 97/20817 (U.S. Pat. No. 5,849,931), WO 99/02500 (U.S. Pat. No. 6,187,930), WO 99/07684 (U.S. Pat. No. 8,118,009) and WO 99,52525 (U.S. Pat. No. 6,410,582) we have also described several procedures to prepare enantiomerically pure compounds with general formula (I).

Still renal colic, is not mentioned in any application and it was quite surprising that general formula (I) compounds, and their physiologically acceptable salts, are especially useful for producing drugs, in human or veterinary therapeutics, to provide a relieve from renal colic with the hope of less side effects than the state of the art.

### DETAILED DESCRIPTION OF THE INVENTION

The present invention refers to a method of treatment of a patient or a mammal, including man, suffering from renal colic characterized in that the method comprises the administration of a therapeutically effective amount of a carbinol compound of general formula (II)

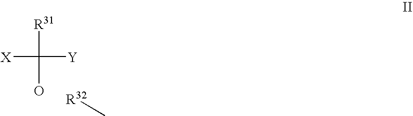

wherein

R³¹ represents a hydrogen atom, a linear or branched alkyl radical, a linear or branched alkenyl radical, an optionally at least mono-substituted cycloaliphatic radical, which may contain at least one nitrogen atom as ring member, or a phenyl radical,

R³² represents a hydrogen atom, an optionally at least one nitrogen atom as ring member containing cycloaliphatic radical, which may be at least mono-substituted by a linear or branched alkyl radical and/or which may be bound via a linear or branched alkylene group, an NR³³R³⁴-moiety, which is bound via a linear or branched alkylene group, or an NR³⁵R³⁵-moiety, which is bound via a linear or branched alkylene group,

R³³ and R³⁴, identical or different, represent a linear or branched alkyl radical or an unsubstituted benzyl radical,

R³⁵ and R³⁶ together with the bridging nitrogen atom represent a saturated, unsubstituted, optionally at least one further heteroatom as ring member containing heterocyclic radical,

X represent an optionally at least mono-substituted phenyl radical or an optionally at least mono-substituted thienyl radical, wherein in each case the substituent may be independently selected from the group consisting of a linear or branched alkyl radical, a linear or branched alkoxy group, a linear or branched alkyl radical, which is at least partially halogenated and a halogen atom,

Y represents a heteroaryl radical, which contains one or more nitrogen atoms as ring members and which is unsubstituted or at least mono-substituted by one or more substitutes independently from one another selected from the group consisting of a halogen atom, a linear or branched alkyl radical, a benzyl radical, a clano group bound via a linear or branched C₁₋₄alkylene group, a carboxy group bound via a linear or branched C₁₋₄alkylene group, a methoxy carbonyl group bound via a linear or branched C₁₋₄alkylene group, a hydroxy group bound via a linear or branched C₁₋₄alkylene group, an amino group bound via a linear or branched C₁₋₄alkylene group, a (C₁₋₄) dialkylamino group bound via a linear or branched C₁₋₄alkylene group, and a cycloaliphatic radical, which contains at least one nitrogen atom as ring member and which is bound via a linear or branched C₁₋₄alkylene group, or Y represents an unsubstituted heteroaryl radical, which contains two nitrogen atoms as ring members and which is condensed with a saturated, one methyl-substituted nitrogen atom as ring member containing cycloaliphatic group,

optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

In the context of this invention, alkyl and cycloalkyl radicals are understood as meaning saturated and unsaturated (but not aromatic), branched, unbranched and cyclic hydrocarbons, which can be unsubstituted or mono- or polysubstituted, in these radicals, C1-2-alkyl represents C1- or C2-alkyl, C1-3-alkyl represents C1-, C2- or C3-alkyl, C1-4-alkyl represent C1-, C2-, C3- or C4-alkyl, CC1-C5-alkyl represents C1, C2, C3, C4, or C5-alkyl, C1-6-alkyl represents C1-, C2-, C3-, C4, C5- of C6-alkyl, C1-7-alkyl represents C1-, C2-, C3-, C4-, C5, C6- of C7-alkyl, C1-8-alkyl represents C1-, C2-, C3-, C4-, C5-, C6-, C7- or C8-alkyl, C1-C10-alkyl represents C1-, C2-, C3-, C4-, C5-, C6-, C7-, C8-, C9-, C10-alkyl and C1-18alkyl represents C1-, C2-, C3-, C4-, C5-, C6-, C7-, C8-, C9-, C10-, C11-, C12-, C13-, C14-, C15-, C16-, C17-, or C18-alkyl. Furthermore, C3-4-cycloalkyl represents C3- or C4-cycloalkyl, C3-5-cycloalkyl represents C3-, C4- or C5-cycloalkyl, C3-6-cycloalkyl represents C3-, C4-, C5-, or C6-cycloalkyl, C3-7-cycloalkyl represents C3-, C4-, C5-, C6- or C7-cycloalkyl, C3-8-cycloalkyl represents C3-, C4-, C5-, C6-, C7-, or C8-cycloalkyl, C4-5- cycloalkyl represents C4- or C5-cycloalkyl, C4-6-cycloalkyl represents C4-, C5- or C6-cycloalkyl, C4-7-cycloalkyl represents C4-, C5-, C6-, or C7-cycloalkyl, C5-6-cycloalkyl represents C5- or C6-cycloalkyl and C5-7-cycloalkyl represents C5-, C6- or C7-cycloalkyl. In respect of cycloalkyl, the term also includes saturated cycloalkyls in which one or 2 carbon atoms are replaced by a heteroatom, S, N or O. However, mono- or polyunsaturated, preferably monounsaturated, cycloalkyls without a heteroatom in the ring also in particular fall under the term cycloalkyl as long as the cycloalkyl is not an aromatic system. The alkyl and cycloalkyl radicals ere preferably methyl, ethyl, vinyl(ethenyl), propyl, allyl(2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentylmethyl, cyclohexyl, cycloheptyl, cyclooctyl, and also adamantly, CHF2, CF3 or CH2OH as well as pyrazolinone, oxopyrazolinone, [1,4]-dioxane or dioxolane.

Here, in connection with alkyl and cycloalkyl—unless expressly defined otherwise—the term substituted in the context of this invention is understood as meaning replacement of at least one hydrogen radical by F, Cl, Br, I, NH2, SH or OH, “polysubstituted” radicals being understood as meaning that the replacement takes effect both on different and on the same atoms several times with the same or different substituent, for example three times on the same C atom, as in the case of CF3, or at different places, as in the case of —CH(OH)—CH═CH—CHCl2. Particularly preferred substituent here are F, Cl and OH. In respect of cycloalkyl, the hydrogen radical can also be replaced by OC1-3-alkyl or C1-3-alkyl (in each case mono- or polysubstituted or unsubstituted), in particular methyl, ethyl, n-propyl, i-propyl, CF3, methoxy or ethoxy.

The term (CH2)3-6 is to be understood as meaning —CH2-CH2-CH2-, —CH2-CH2-C2-CH2-, —CH2-CH2-CH2-CH2-CH2- and —CH2-CH2-CH2-CH2-CH2-CH2-, (CH2)1-4 is to be understood as meaning —CH2-, —CH2-CH2-, —CH2-CH2-CH2-, and —CH2- CH2-CH2-CH2-, (CH2)4-5 is to be understood as meaning —CH2-CH2-CH2-CH2-, and —CH2- CH2-CH2-CH2- CH2—, etc.

The term “lower alkyl group from C₁ to C₄” (which is equivalent to “lower C_{(1-4)”}Alkyl”) represents a linear or branched chain radical derived from a saturated hydrocarbon of 1 to 4 carbon atoms, such as methyl, ethyl, propyl, isopropyl, butyl, isobutyl, sec-butyl and tert-butyl.

The term “dialkyl(C₁-C₄)aminoalkyl (C₂-C₃), or azaheterocyclyalkyl (C₂-C₃)” represents an alkyl radical with two or three carbon atoms joined to a dialkyl(C₁-C₄)amine or to a cyclic amine, such as, for example, dimethylaminoethyl, dimethylaminopropyl, diethylaminoethyl, piperidinylethyl, morpholinylpropyl, pirrolidinylalkyl, etc.

An aryl radical is understood as meaning ring systems with at least on aromatic ring but without heteroatoms even in only one of the rings. Examples are phenyl, naphthyl, fluoranthenyl, fluorenyl, tetralinyl or indanyl, in particular 9H-fluorenyl or anthracenyl radicals, which can be unsubstituted or monosubstituted or polysubstituted.

A heteroaryl radical is understood as meaning heterocyclic ring systems which have at least one unsaturated ring and can contain one or more heteroatoms from the group consisting of nitrogen, oxygen and/or sulfur and can also be mono or polysubstituted. Examples which may be mentioned from the group of heteroaryls are furan, benzofuran, thiophene. benzothiophene, pyrrole, pyridine, pyrimidine, pyrazine, quionoline, isoquinoline, phthalazine, benzo-1,2,6-thiadiazole, benzothiazole, indole, benzotriazole, benzodioxolane, benzodioxane, carbazole and quinazoline.

Here, in connection with aryl and heteroaryl, substituted is understood as meaning substitution of the aryl or heteroaryl by R, OR, a halogen, preferably F and/or Cl, a CF3, a CN, an NO2, an NRR, a C1-6-alkyl (saturated), a C1-1-alkoxy, a C3-8-cycloalkoxy, a C3-6-cycloalkyl or a C2-6-alkylene.

The compounds of general formula (II) (as well as I, Ia, Ib and Ic) can be synthesised according to the procedures described in patents EP 289380, U.S. Pat. No. 5,017,596 or WO 99/52525. The compounds of general formula (II) (as well as I, Ia, Ib and Ic) have a stereogenic centre and the invention refers both to the use of a pure enantiomer and to the use of a mixture of enantiomers. The enantiomers can be prepared by any of the procedures described in our patents WO 97/20817 (U.S. Pat. No. 5,849,931), WO 99/02500 (U.S. Pat. No. 6,187,930), WO 99/07684 (U.S. Pat. No. 6,118,009) and WO 99152525 (U.S. Pat. No. 6,410,582).

In the context of this invention the term “(dimethylamino)” shall be treated and considered absolutely identical to the term “(dimethylamine). The selection of the first term to describe compounds was only due a seeming more fitting chemical nomenclature.

In a preferred aspect of the invention the method is characterized in that R³¹ represents a hydrogen atom, a linear or branched C₁₋₄ alkyl radical, a linear or branched C₂₋₄ alkenyl radical, a 5- or 6-membered cycloaliphatio radical, which may contain at least one nitrogen atom as ring member and/or which may be at least mono-substituted by a linear or branched C₁₋₄ alkyl radical, or a phenyl radical, preferably a hydrogen atom, a linear or branched C₁₋₄ alkyl radical, a vinyl group, a cyclohexyl radical, an N-Methyl-piperidyl radical or a phenyl radical.

In a preferred aspect of the invention the method is characterized in that R³² represents a hydrogen atom, an optionally at least one nitrogen atom as ring member containing, 5 or 6-membered cycloaliphatic radical, which may be at least mono-substituted by a linear or branched C₁₋₄-alkyl radical and/or which may be bound via a linear or branched C₁₋₄-alkylene group, a NR³³R³⁴-moiety, which is bound via a linear or branched C₁₋₄ alkylene group, or a NR³⁵R³⁶-moiety, which is bound via a linear or branched C₁₋₄ alkylene group, preferably a hydrogen atom, an optionally at least one nitrogen atom as ring member containing, 5- or 6-membered cycloaliphatic radical, which may be at least mono-substituted by a linear or branched C₁₋₄-alkyl radical and/or which may be bound via a linear or branched C₁₋₄-alkylene group, a NR³³R³⁴-moiety, which is bound via a linear or branched C₂₋₃ alkylene group, or a NR³⁵R³⁵-moiety, which is bound via a linear or branched C₂₋₃ alkylene group.

In a preferred aspect of the invention the method is characterized in that R³³ and R³⁴, identical or different, independently from one another represent a linear or branched C₁₋₄ alkyl radical or en unsubstituted benzyl radical, preferably a linear or branched C₁₋₄ alkyl radical.

In a preferred aspect of the invention the method is characterized in that R³⁵ and R³⁶ together with the bridging nitrogen atom represent a saturated, unsubstituted, optionally at least one oxygen atom as ring member containing, 5 or 6-membered heterocyclic radical.

In a preferred aspect of the invention the method is characterized in that X represents an optionally at least mono-substituted phenyl radical or an optionally at least mono-substituted thienyl radical, wherein in each case the substituent may be independently selected from the group consisting of a linear or branched C₁₋₄ alkyl a radical, a linear or branched C₁₋₄ alkoxy radical, a linear or brand C₁₋₄ alkyl radical, which is at least partially fluorinated, a fluorine atom, a chlorine atom and a bromine atom, preferably represents en optionally at least mono-substituted phenyl radical or an optionally at least mono-substituted thienyl radical, wherein in each case the substituted may be independently selected from the group consisting of a methyl radical, a methoxy radical, a trifluoromethyl radical, a fluorine atom, a chlorine atom and a bromine atom.

In a preferred aspect of the invention the method is characterized in that Y represents an azole radical selected from the group consisting of

a) a pyrazole of the general formula (a):

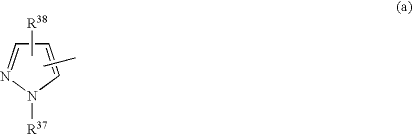

in which R³⁷ represents a linear or branched C₁₋₁₂ alkyl radical, a benzyl radical or a radical of the type;

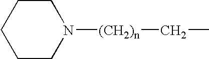

in which n=1 or 2, and

R³⁸ represents a hydrogen atom, a methyl radical or a halogen atom, preferably a hydrogen atom, a methyl radical, a bromine atom or a chlorine atom,

b) an imidazole of the general formula

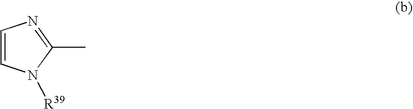

in which R³⁹ represents a hydrogen atom, a C₁₋₁₂ alkyl radical, a benzyl radical, or a radical of the general formula (b1):

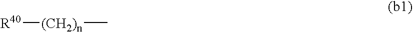

in which n is 2, 3 or 4 and R⁴⁰ represents a piperidinyl radical, a phenyl radical, a cyano group, a hydroxyl radical, a carboxy radical, an amino group, a dimethylamino group, or a methyl ester (CH₃—O—C(═O)—) group, and

(c) an imidazole of the following formula:

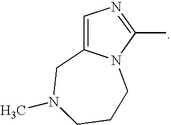

in a preferred aspect of the invention the method is characterized in that at least the one administered compound is a carbinol compound of general formula II

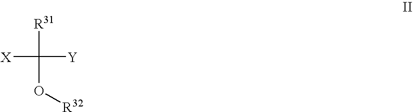

is present, wherein

R³¹ represents a hydrogen atom, a methyl radical, an ethyl radical, an n-propyl radical, an isopropyl radical, a sec-butyl radical, a tert-butyl radical, an n-butyl radical, a vinyl radical, a cyclohexyl radical, an N-ethyl-piperidinyl group, or a phenyl group,

R³² represents a hydrogen atom, a dimethylaminoethyl group, a pyrrolidinylethyl group, a piperidinylethyl group, a methyl-benzyl-aminoethyl group, a morpholinylethyl group, a diisopropylaminoethyl group, a dimethylaminopropyl group, a piperidinylpropyl group, a pyrrolidinylpropyl group, a morpholinylpropyl group, an N-methyl-2-piperidyl group, an N-ethyl-2-piperidyl group, an N-propyl-2-piperidyl group, an N-methyl-2-pyrrolidinyl group, an N-ethyl-2-pyrrolidinyl group, an N-propyl-2-pyrrolidinyl group, or a 2-dimethylaminoethyl-1-methyl group,

X represents a phenyl radical, a 2-methyl-phenyl radical, a 3-methyl-phenyl radical a 4-methyl phenyl radical, a 2-chloro-phenyl radical, a 3-chloro-phenyl radical, a 4-chloro-phenyl radical, a 2-fluoro-phenyl radical, a 3-fluoro-phenyl radical, a 4-fluoro-phenyl radical, a 2-trifluoromethyl-phenyl radical, a 3-trifluoromethyl-phenyl radical, a 4-trifluoromethyl-phenyl radical, a 2-methoxy-phenyl radical, a 3-methoxy-phenyl radical, a 4-methoxy-phenyl radical, a 3,4,5-tris-methoxy phenyl radical, a 3,4-dichloro-phenyl radical, a 2,4-dichloro-phenyl radical, a thien-2-yl radical, a thien-3-yl radical, a 3-methyl-thien-2-yl radical, a 5-methyl-thien-2-yl-radical, a 5-bromo-thien-2-yl radical or a 4-bromo-thien-2-yl-radical,

Y represents an azole radical selected from the group consisting of

a) a pyrazole of the general formula (a):

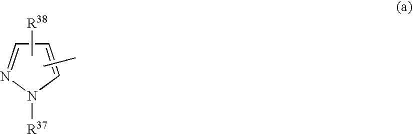

in which

R³⁷ represents a methyl radical, an ethyl radical, an n-propyl radical, an iso-propyl radical, an n-butyl radical, a sec-butyl radical or a tert-butyl radical,

R³⁸ represents a hydrogen atom, a methyl radical, a bromine atom or a chlorine atom,

b) an imidazole of the general formula

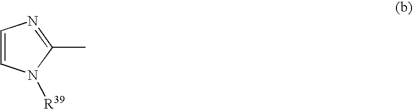

in which R³⁹ represents a hydrogen atom, a methyl radical, an ethyl radical, an n-propyl radical, an iso-butyl radical, an n-butyl radical, a sec-butyl radical a tert-butyl radical, an n-pentyl radical, an n-hexyl radical, an n-heptyl radical, an n-octyl radical, an n-nonyl radical, an n-decyl radical, an n-undecyl radical an n-dodecyl radical, a benzyl radical, or a radical of the general formula (b1):
R⁴⁰—(CH₂)ₙ— (b1)

in which n is 2, 3 or 4 and R⁴⁰ represents a piperidinyl radical a phenyl radical, a cyano group, hydroxyl radical, a carboxy radical, an amino group, a dimethylamino group, or a methyl ester group, and

(c) an imidazole of the following formula:

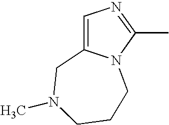

optionally in form of one of its stereoisomers, preferably enantiomers or diastereomers, its racemate or in form of a mixture of at least two of its stereoisomers, preferably enantiomers and/or diastereomers, in any mixing ratio, or a corresponding salt thereof, or a corresponding solvate.

In a preferred aspect of the invention the method is characterised in that at least one administered component is selected from the group consisting of

[1] 2-{α-[2-(dimethylamino)ethoxy]benzyl}-1-methyl-1H-imidazole,

[2] 2-{4-chloro-α-[2-(dimethylamino)ethoxy]-methylbenzyl}-1-methyl-1H-imidazole,

[3] 2-{4-chloro-α-[2-(dimethylamino)ethoxy]benzyl}-1-methyl-1H-imidazole,

[4] 2-{3-chloro-α-[2-(dimethylamino)ethoxy]benzyl}-1-methyl-1H-imidazole,

[5] 2-{4-chloro-α-[2-(dimethylamino)ethoxy]-α-methylbenzyl}-1-methyl-1H-imidazole,

[6] 2-{4-fluoro-α-[2-(dimethylamino)ethoxy]-α-methylbenzyl}-1-methyl-1H-imidazole,

[7] 2-{α-[2-(dimethylamino)ethoxy]-α-methyl-3-(trifluoromethyl)benzyl}-1-methyl-1H-imidazole,

[8] 2-{3-chloro-α-[2-(dimethylamino)ethoxy]-α-methylbenzyl}-1-methyl-1H-imidazole,

[9] 2-{3-chloro-α-[2-(dimethylamino)ethoxy]-α-propylbenzyl}-1-methyl-1H-imidazole,

[10] 1-butyl-2-{4-chloro-α-[2-(dimethylamino)ethoxy]-α-methylbenzyl}-1H-imidazole,

[11] 2-{α-[2-(dimethylamino)ethoxy]-α-methyl-4-methoxybenzyl}-1-methyl-1H-imidazole,

[12] 2-{3-chloro-α-methyl-α-[2-(N-pyrrolidinyl)ethoxy]benzyl}-1-methyl-1H-imidazole,

[13] 2-{α-[2-(dimethylamino)ethoxy]-α-propyl-3,4,5-trimethoxybenzyl}-1-dodecyl-1H-imidazole,

[14] 1-butyl-2-{α-[2-(dimethylamino)ethoxy]-4-(trifluoromethyl)benzyl}-1H-imidazole,

[15] 1-methyl-2-{α-[2-methyl-α-[2-(N-piperidyl)ethoxy]-3-(trifluoromethyl)benzyl}-1H-imidazole,

[16] 2-{α-cyclohexyl-3,4-dichloro-α-[2-(dimethylamino)ethoxy]benzyl}-1-methyl-1H-imidazole,

[17] 2-{3,4-dichloro-α-[2-(dimethylamino)ethoxy]-α-propylbenzyl}-1-methyl-1H-imidazole,

[18] 2-{3,4-dichloro-α-[2-(dimethylamino)ethoxy]-α-methylbenzyl}-1-methyl-1H-imidazole,

[19] 2-{3,4-dichloro-α-[2-(dimethylamino)ethoxy]benzyl}-1-methyl-1H-imidazole,

[20] 2-{4-chloro-α-[2-(dimethylamino)ethoxy]-α-methylbenzyl}-1-[2-(N-piperidyl)ethyl}-1H-imidazole,

[21] 2-{4-chloro-α-[2-(dimethylamino)ethoxy]-α-methylbenzyl}-1-[2-(N-piperidyl)propyl}-1H-imidazole,

[22] 1-(3-cyanopropyl)-2-{4-chloro-α-[2-(dimethylamino)ethoxy]benzyl}-1H-imidazole,

[23] 2-{4-chloro-α-[2-(dimethylamino)ethoxy]-α-(N-methyl-4-piperidyl)benzyl}-1-methyl-1H-imidazole,

[24] 1-benzyl-2-{α-[(N-benzyl-N-methylamino)ethoxy]-4-chlorobenzyl}-1H-imidazole,

[25] 2-{4-chloro-α-[2-(dimethylamino)ethoxy]-α-methylbenzyl}-7-methyl-6,7,8,9-tetrahydro-1H-imidazole[1,5-a][1,4]diazepine,

[26] 2-{4-chloro-α-[2-(dimethylamino)ethoxy]benzyl}-7-methyl-6,7,8,9-tetrahydro-1H-imidazole[1,5-a][1,4]diazepine,

[27] 1-butyl-5-{α-[2-(dimethylamino)ethoxy]benzyl}-1H-pyrazole,

[28] 5-{α-(4-chlorophenyl)-α-[2-(dimethylamino)ethoxy]benzyl}-1-methyl-1H-pyrazole,

[29] 1-butyl-5-{α-[2-(dimethylamino)ethoxy]-3,4 5-trimethoxybenzyl}-1H-pyrazole

[30] 1-butyl-5-{4-chloro-α-[2-(dimethylamino)ethoxy]-α-methylbenzyl}-1H-pyrazole,

[31] 5-{α-[2-(dimethylamino)ethoxy]benzyl}-1-methyl-1H-pyrazole,

[32] 5-{α-[2-(dimethylamino)ethoxy]-α-methylbenzyl}-1-methyl-1H-pyrazole,

[33] 5-{α-[2-(dimethylamino)ethoxy]-3,4,5-trimethoxybenzyl}-1-methyl-1H-pyrazole,

[34] 1-methyl-5-{α-[2-(N-pyrrolidinyl)ethoxy]benzyl}-1H-pyrazole,

[35] 1-methyl-5-{α-[2-(N-morpholinyl)ethoxy]benzyl}-1H-pyrazole,

[36] 5-{α-[2-(dimethylamino)ethoxy]-α-methyl-3,4,5-trimethoxybenzyl}-1-methyl-1H-pyrazole,

[37] 4-bromo-5-{α-[2-(dimethylamino)ethoxy]benzyl}-1-methyl-1H-pyrazole,

[38] 1,3-dimethyl-5-{α-[2-(dimethylamino)ethoxy]-α-methylbenzyl}-1H-pyrazole,

[39] 1,3-dimethyl-5-{α-[2-(dimethylamino)ethoxy]benzyl}-1H-pyrazole,

[40] 5-{α-[2-(dimethylamino)ethoxy]-2-methylbenzyl}-1-methyl-1H-pyrazole,

[41] 4-chloro-5-{4-chloro-α-[2-(dimethylamino)ethoxy]benzyl}-1-methyl-1H-pyrazole,

[42] 5-{4-chloro-α-[2-(dimethylamino)ethoxy]benzyl}-1-methyl-1H-pyrazole,

[43] 5-{3-chloro-α-[2-(dimethylamino)ethoxy]benzyl}-1-methyl-1H-pyrazole,

[44] 5-{α-[2-(dimethylamino)ethoxy]-4-methylbenzyl}-1-methyl-1H-pyrazole,

[45] 5-{2-chloro-α-[2-(dimethylamino)ethoxy]benzyl}-1-methyl-1H-pyrazole,

[46] 1-methyl-5-{α-[2-(N-piperidyl)ethoxy]benzyl}-1H-pyrazole,

[47] 1-methyl-5-{α-[2-(N-propyl-2-piperidyl)ethoxy]benzyl}-1H-pyrazole,

[48] 5-{α-[2-(N-ethyl-2-piperidyl)ethoxy]benzyl}-1-methyl-1H-pyrazole,

[49] 1-methyl-5-{α-[2-(N-methyl-2-pyrrolidinyl)ethoxy]benzyl}-1H-pyrazole,

[50] 5-{α-[2-(diisopropylamine)ethoxy]benzyl}-1-methyl-1H-pyrazole,

[51] 1-methyl-5-{α-[2-(N-methyl-2-piperidyl)ethoxy]benzyl}-1H-pyrazole,

[52] 2-{4-chloro-α-[3-(dimethylamino)propoxy]-α-methylbenzyl}-1-methyl-1H-imidazole,

[53] 2-{3-chloro-α-[3-(dimethylamino)propoxy]benzyl}-1-methyl-1H-imidazole,

[54] 2-{4-chloro-α-[3-(dimethylamino)propoxy]-α-ethylbenzyl}-1-methyl-1H-imidazole,

[55] 2-{α-butyl-3-chloro-α-[3-(dimethylamino)propoxy]benzyl}-1-methyl-1H-imidazole,

[56] 2-{α-cyclohexyl-4-chloro-α-[3-(dimethylamino)propoxy]benzyl}-1-methyl-1H-imidazole,

[57] 2-{α-[3-(dimethylamino)propoxy]-4-fluoro-α-methylbenzyl}-1-methyl-1H-imidazole,

[58] 2-{α-[3-(dimethylamino)propoxy]-α-methyl-3-(trifluoromethyl)benzyl}-1-methyl-1H-imidazole,

[59] 2-{2-chloro-α-[3-(dimethylamino)propoxy]-α-methylbenzyl}-1-methyl-1H-imidazole,

[60] 2-{3-chloro-α-[3-(dimethylamino)propoxy]-α-methylbenzyl}-1-methyl-1H-imidazole,

[61] 2-{α-[3-(dimethylamino)propoxy]-α-methyl-3,4,5-trimethoxybenzyl}-1-methyl-1H-imidazole,

[62] 2-{α-[3-(dimethylamino)propoxy]-α-methyl-4-methoxybenzyl}-1-methyl-1H-imidazole,

[63] 2-{4-chloro-α-[3-(dimethylamino)propoxy]benzyl}-1-methyl-1H-imidazole,

[64] 2-{α-[3-(dimethylamino)propoxy]-3,4,5-trimethoxybenzyl}-1-methyl-1H-imidazole,

[65] 2-{α-[3-(dimethylamino)propoxy]-α-methyl-4-(trifluoromethyl)benzyl}-1-methyl-1H-imidazole,

[66] 2-{α-[3-(dimethylamino)propoxy]-3-(trifluoromethyl)benzyl}-1-methyl-1H-imidazole,

[67] 2-{α-[3-(dimethylamino)propoxy]-4-(trifluoromethyl)benzyl}-1-methyl-1H-imidazole,

[68] 2-{α-[3-(dimethylamino)propoxy]-4-methoxybenzyl}-1-methyl-1H-imidazole,

[69] 2-{α-[3-(dimethylamino)propoxy]-3-(trifluoromethyl)benzyl}-1-methyl-1H-imidazole,

[70] 1-butyl-2-{4-chloro-α-[3-(dimethylamino)propoxy]-α-methylbenzyl}-1H-imidazole,

[71] 1-butyl-2-{α-butyl-α-[3-(dimethylamino)propoxy]-3,4,5-trimethoxybenzyl}-1H-imidazole,

[72] 1-butyl-2-{α-butyl-2-chloro-α-[3-(dimethylamino)propoxy]benzyl}-1H-imidazole

[73] 1-butyl-2-{α-butyl-2,4-dichloro-α-[3-(dimethylamino)propoxy]benzyl}-1H-imidazole,

[74] 1-butyl-2-{α-[3-(dimethylamino)propoxy]-4-(trifluoromethyl)benzyl}-1H-imidazole,

[75] 2-{4-chloro-α-[3-(N-piperidyl)propoxy]benzyl}-1-methyl-1H-imidazole,

[76] 1-methyl-2-{α-methyl-α-[3-(N-piperidyl)propoxy]-4-trifluoromethyl)benzyl}-1H-imidazole,

[77] 2-{α-butyl-2-chloro-α-[3-(dimethylamino)propoxy]benzyl}-1-methyl-1H-imidazole,

[78] 2-{α-butyl-3,4-dichloro-α-[3-(dimethylamino)propoxy]benzyl}-1-methyl-1H-imidazole,

[79] 2-{3,4-dichloro-α-[3-(dimethylamino)propoxy]-α-methylbenzyl}-1-methyl-1H-imidazole,

[80] 2-{3,4-dichloro-α-[3-(dimethylamino)propoxy]benzyl}-1-methyl-1H-imidazole,

[81] 2-{α-cyclohexyl-3,4-dichloro-α-[3-(dimethylamino)propoxy]benzyl}-1-methyl-1H-imidazole,

[82] 2-{4-chloro-α-[3-(dimethylamino)propoxy]-α-methylbenzyl}-α-[2-(N-piperidyl)ethyl]-1H-imidazole,

[83] 2-{4-chloro-α-[3-(dimethylamino)propoxy]-α-methylbenzyl}-1-[2-(N-piperidyl)propyl]-1H-imidazole,

[84] 2-{4-chloro-α-[3-(dimethylamino)propoxy]-α-(N-methyl-4-piperidyl)benzyl}-1-methyl-1H-imidazole,

[85] 1-butyl-5-{α-[3-(dimethylamino)propoxy]benzyl}-1H-imidazole,

[86] 1-butyl-5-{4-chloro-α-[3-(dimethylamino)propoxy]-α-methylbenzyl}-1H-pyrazole,

[87] 5-{α-[3-(dimethylamino)propoxy]benzyl}-1-methyl-1H-pyrazole,

[88] 5-{α-[3-(dimethylamino)propoxy]-α-methylbenzyl}-1-methyl-1H-pyrazole,

[89] 1,3-dimethyl-5-{α-[3-(dimethylamino)propoxy]-α-methylbenzyl}-1H-pyrazole ,

[90] 1,3-dimethyl-5-{α-[3-(dimethylamino)propoxy]benzyl}-1H-pyrazole,

[91] 5-{α-[3-(dimethylamino)propoxy]-2-methylbenzyl}-methyl-1H-pyrazole,

[92] 5-chloro-5-{4-chloro-α-[3-(dimethylamino)propoxy]benzyl}-1-methyl-1H-pyrazole,

[93] 1-methyl-5-{α-[3-(dimethylamino)propoxy]benzyl}-1H-pyrazole,

[94] 1-methyl-5-{α-[3-(dimethylamino)propoxy]benzyl}-1H-pyrazole,

[95] 4-{4-chloro-α-[2-(dimethylamino)ethoxy]benzyl}-1-methyl-1H-pyrazole,

[96] 4-{4-chloro-α-[2-(dimethylamino)ethoxy]-α-methylbenzyl}-1-methyl-1H-pyrazole,

[97] 4-{4-chloro-α-[2-(N-propyl-2-piperidyl)ethoxy]benzyl}-1-methyl-1H-pyrazole,

[98] 4-{4-chloro-α-[2-(N-methyl-2-piperidyl)ethoxy]benzyl}-1-methyl-1H-pyrazole,

[99] 4-{4-chloro-α-[2-(N-ethyl-2-piperidyl)ethoxy]benzyl}-1-methyl-1H-pyrazole,

[100] 4-{4-chloro-α-[2-(diisopropylamino)ethoxy]benzyl}-1-methyl-1H-pyrazole,

[101] 4-{4-chloro-α-[2-(N-methyl-2-pyridinyl)ethoxy]benzyl}-1-methyl-1H-pyrazole,

[102] 4-{α-[3-(dimethylamino)propoxy]benzyl}-1-methyl-1H-pyrazole,

[103] 4-{4-chloro-α-[3-(N-morpholinyl)propoxy]benzyl}-1-methyl-1H-pyrazole,

[104] 4-{4-chloro-α-[3-(N-pyrrolidinyl)propoxy]benzyl}-1-methyl-1H-pyrazole,

[105] 2-(α-hydroxybenzyl)-1H-imidazole,

[106] 2-(4-chloro-α-hydroxybenzyl)-1H-imidazole,

[107] 2-(4-chloro-α-hydroxybenzyl)-1-methyl-1H-imidazole,

[108] 2-(3-chloro-α-hydroxybenzyl)-1-methyl-1H-imidazole,

[109] 2-(4-fluoro-α-hydroxybenzyl)-1-methyl-1H-imidazole,

[110] 2-[α-hydroxy-3-(trifluoromethyl)benzyl]-1-methyl-1H-imidazole,

[111] 2-[α-hydroxy-4-(trifluoromethyl)benzyl]-1-methyl-1H-imidazole,

[112] 2-(α-hydroxy-3,4,5-trimethoxybenzyl)-1-methyl-1H-imidazole,

[113] 2-(3,4-dichloro-α-hydroxybenzyl)-1-methyl-1H-imidazole,

[114] 1-butyl-2-[α-hydroxy-4-(trifluoromethyl)benzyl]-1H-imidazole,

[115] 1-butyl-2-(3,4-dichloro-α-hydroxybenzyl)-1H-imidazole,

[116] 1-butyl-2-(4-chloro-α-hydroxybenzyl)-1H-imidazole,

[117] 1-butyl-2-(α-hydroxy-3,4,5-trimethoxybenzyl)-1H-imidazole,

[118] 1-dodecyl-2-(α-hydroxy-3,4,5trimethoxybenzyl)-1H-imidazole,

[119] 2-(α-butyl-3-chloro-α-hydroxybenzyl)-1-methyl-1H-imidazole,

[120] 2-(3-chloro-α-hydroxy-α-methylbenzyl)-1-methyl-1H-imidazole,

[121] 2-(4-chloro-α-hydroxy-α-methylbenzyl)-1-methyl-1H-imidazole,

[122] 2-[4-chloro-α-hydroxy-α-(N-methyl-4-piperidyl)benzyl]-1-methyl-1H-imidazole,

[123] 2-(4-chloro-α-ethyl-α-hydroxybenzyl)-1-methyl-1H-imidazole,

[124] 2-(α-butyl-4-chloro-α-hydroxybenzyl)-1-methyl-1H-imidazole,

[125] 2-(α-cyclohexyl-4-chloro-α-hydroxybenzyl)-1-methyl-1H-imidazole,

[126] 2-(2-chloro-α-hydroxy-α-methylbenzyl)-1-methyl-1H-imidazole,

[127] 2-(α-butyl-2-chloro-α-hydroxybenzyl)-1-methyl-1H-imidazole,

[128] 2-[α-hydroxy-α-methyl-3-(trifluoromethyl)benzyl]-1-methyl-1H-imidazole,

[129] 2-[α-butyl-α-hydroxy-3-(trifluoromethyl)benzyl]-1-methyl-1H-imidazole,

[130] 2-[α-cyclohexyl-α-hydroxy-3-(trifluoromethyl)benzyl]-1-methyl-1H-imidazole,

[131] 2-[α-hydroxy-α-methyl-4-(trifluoromethyl)benzyl]-1-methyl-1H-imidazole,

[132] 2-(4-fluoro-α-hydroxy-α-methylbenzyl)-1H-imidazole,

[133] 2-(α-hydroxy-α-methyl-4-methoxybenzyl)-1-methyl-1H-imidazole,

[134] 2-(3,4-dichloro-α-hydroxy-α-methylbenzyl)-1-methyl-1H-imidazole,

[135] 2-(α-butyl-3,4-dichloro-α-hydroxybenzyl)-1-methyl-1H-imidazole,

[136] 2-(α-cyclohexyl-3,4-dichloro-α-hydroxybenzyl)-1-methyl-1H-imidazole,

[137] 2-(α-hydroxy-α-methyl-3,4,6-trimethoxybenzyl)-1-methyl-1H-imidazole,

[138] 1-butyl-2-(4-chloro-α-hydroxy-α-methylbenzyl)-1H-imidazole,

[139] 1-butyl-2-(α-butyl-4-chloro-α-hydroxybenzyl)-1H-imidazole,

[140] 1-butyl-2-[4-chloro-α-hydroxy-α-(N-methyl-4-piperidyl)benzyl]-1H-imidazole,

[141] 1-butyl-2-(α-butyl-α-hydroxy-3,4,5-trimethoxybenzyl)-1H-imidazole,

[142] 1-butyl-2-(α-butyl-2-chloro-α-hydroxybenzyl)-1H-imidazole,

[143] 1-butyl-2-[α-ethyl-α-hydroxy-3-trifluoromethyl)benzyl]-1H-imidazole,

[144] 1-butyl-2-(α-butyl-2,4-dichloro-α-hydroxybenzyl)-1H-imidazole,

[145] 2-(4-chloro-α-hydroxy-α-methylbenzyl)-1-[2-(N-piperidyl)ethyl]-1H-imidazole,

[146] 2-(4-chloro-α-hydroxy-α-methylbenzyl)-1-(3-dimethylaminopropyl)-1H-imidazole,

[147] 2-(α-butyl-α-hydroxy-3,4,5-trimethoxybenzyl)-1-dodecyl-1H-imidazole,

[148] 1-benzyl-2-[α-butyl-α-hydroxy-3-(trifluoromethyl)benzyl]-1H-imidazole,

[149] 1-benzyl-2-(4-chloro-α-hydroxy-α-methylbenzyl)-1H-imidazole,

[150] 1-(2-cyanoethyl)-2-(4-chloro-α-hydroxybenzyl)-1H-imidazole

[151] 1-(3-aminopropyl)-2-(4-chloro-α-hydroxybenzyl)-1H-imidazole,

[152] 3-[2-(3-chloro-α-hydroxybenzyl)-1H-imidazole-1-yl]propanoic aid,

[153] 2-(4-chloro-α-hydroxybenzyl)-1-(3-hydroxypropyl)-1H-imidazole,

[154] 3-[2-(3-chloro-α-hydroxybenzyl)-1H-imidazole-1-yl]methyl-propanoate,

[155] 2-(α-hydroxybenzyl)-1-(3hydroxypropyl)-1H-imidazole,

[156] 2-(α-hydroxy-4-methylbenzyl)-1-(3-hydroxypropyl)-1H-imidazole,

[157] 2-(α-hydroxy-4-methoxylbenzyl)-1-(3-hydroxypropyl)-1H-imidazole,

[158] 2-(3,4-dichloro-α-hydroxybenzyl)-1-(3-hydroxypropyl)-1H-imidazole,

[159] 3-{2-(α-hydroxybenzyl)-1H-imidazole-yl}-methyl propanoate,

[160] 2-(4-chloro-α-hydroxybenzyl)-1-(4-hydroxybutyl)-1H-imidazole,

[161] 1-(3-cyanopropyl)-2-(4-chloro-α-hydroxybenzyl)-1H-imidazole,

[162] 4-[2-(4-chloro-α-hydroxybenzyl)-1H-imidazole-1-yl]butanoic acid.

[163] 4-[2-(4-chloro-α-hydroxybenzyl)-1H-imidazole-1-yl]-methyl butanoate.

[164] 1-butyl-5-(α-hydroxybenzyl)-1H-pyrazole,

[165] 5-(4-chloro-α-hydroxybenzyl)-1-methyl-1H-pyrazole,

[166] 5-(α-hydroxy-3,4,5-trimethoxybenzyl)-1-methyl-1H-pyrazole,

[167] 1-butyl-5-(α-hydroxy-3,4,5-trimethoxybenzyl)-1H-pyrazole,

[168] 4-bromo-5-(α-hydroxybenzyl)-1-methyl-1H-pyrazole,

[169] 5-[α-(4-chlorophenyl)-α-hydroxybenzyl]-1-methyl-1H-pyrazole,

[170] 1-butyl-5-(4-chloro-α-hydroxy-α-methylbenzyl)-1H-pyrazole,

[171] 5-(α-hydroxy-α-methylbenzyl)-1-methyl-1H-pyrazole,

[172] 5-(α-hydroxy-α-methyl-3,4,5-trimethoxybenzyl)-1-methyl-1H-pyrazole,

[173] 1,3-dimethyl-5-(α-hydroxy-α-methylbenzyl)-1H-pyrazole,

[174] 1-butyl-5-(α-hydroxy-α-vinylbenzyl)-1H-pyrazole,

[175] 1-butyl-5-(4-chloro-α-hydroxy-α-vinylbenzyl)-1H-pyrazole,

[176] 4-chloro-5-(α-hydroxybenzyl)-1-methyl-1H-pyrazole,

[177] 5-(α-hydroxy-2-methylbenzyl)-1-methyl-1H-pyrazole,

[178] 5-(3-chloro-α-hydroxybenzyl)-1-methyl-1H-pyrazole,

[179] 5-(α-hydroxy-4-methylbenzyl)-1-methyl-1H-pyrazole,

[180] 5-(2-chloro-α-hydroxybenzyl)-1-methyl-1H-pyrazole,

[181] 5-(α-hydroxy-4-methoxybenzyl)-1-methyl-1H-pyrazole,

[182] 5-{α-[2-(dimethylamino)ethoxy]-2-thienylmethyl}-1-methyl-1H-pyrazole,

[183] 5-{α-[2-(dimethylamino)ethoxy]-2-thienylmethyl}-1-methyl-1H-pyrazole citrate,

[184] 5-{α-[2-(dimethylamino)ethoxy]-3-thienylmethyl}-1-methyl-1H-pyrazole,

[185] 2-{α-[2-(dimethylamino)ethoxy]-2-thienylmethyl}-1-methyl-1H-imidazole,

[186] 5-{α-[2-(dimethylamino)ethoxy]-3-methyl-2-thienylmethyl}-1-methyl-1H-pyrazole,

[187] 5-{α-[2-(dimethylamino)ethoxy]-5-methyl-2-thienylmethyl}-1-methyl-1H-pyrazole,

[188] 5-{5-bromo-α-[2-(dimethylamino)ethoxy]-2-thienylmethyl}-1-methyl-1H-pyrazole,

[189] 5-{4-bromo-α-[2-(dimethylamino)ethoxy]-2-thienylmethyl}-1-methyl-1H-pyrazole,

[190] 5-{α-[2-(dimethylamino)ethoxy]-α-methyl-2-thienylmethyl}-1-methyl-1H-pyrazole,

[191] 5-{α-[2-(dimethylamino)ethoxy]benzyl}-1-methyl-1H-pyrazole citrate,

[192] (±)-5-{α-[2-(dimethylamino)-1-(methyl)ethoxy]benzyl}-1-methyl-1H-pyrazole,

[193] (±)-5-{α-[2-(dimethylamino)-1-(methyl)ethoxy]benzyl}-1-methyl-1H-pyrazole,

[194] (+)-5-{α-[2-(dimethylamino)ethoxy]-2-thienylmethyl}-1-methyl-1H-pyrazole,

[195] (−)-5-{α-[2-(dimethylamino)ethoxy]-2-thienylmethyl}-1-methyl-1H-pyrazole,

[196] (+)-5-{α-[2-(dimethylamino)ethoxy]-2-thienylmethyl}-1-methyl-1H-pyrazole citrate,

[197] (−)-5-{α-[2-(dimethylamino)ethoxy]-2-thienylmethyl}-1-methyl-1H-pyrazole citrate,

[198] (+)-5-{α-[2-(dimethylamino)ethoxy]-2-thienylmethyl}-1-methyl-1H-pyrazole-D-ditoluyitartrat,

[199] (−)-5-{α-[2-(dimethylamino)ethoxy]-2-thienylmethyl}-1-methyl-1H-pyrazole D-ditoluyitartrat,

[200] (+)-5-{α-[2-(dimethylamino)ethoxy]benzyl}-1-methyl-1H-pyrazole citrate,

[201] (−)-5-{α-[2-(dimethylamino)ethoxy]benzyl}-1-methyl-1H-pyrazole citrate,

[202] 5-(α-hydroxy-2-thienylmethyl)-1-methyl-1H-pyrazole,

[203] 5-(α-hydroxy-3-methyl-2-thienylmethyl)-1-methyl-1H-pyrazole,

[204] 5-(α-hydroxy-5-methyl-2-thienylmethyl)-1-methyl-1H-pyrazole,

[205] 5-(5-bromo-α-hydroxy-2-thienylmethyl)-1-methyl-1H-pyrazole,

[206] 5-(4-bromo-α-hydroxy-2-thienylmethyl)-1-methyl-1H-pyrazole and

[207] 5-(α-hydroxy-α-methyl-2-thienylmethyl)-1-methyl-1H-pyrazole.

Another aspect of the invention is a method of treatment of a patient or a mammal, including man, suffering from renal colic characterized in that the method comprises the administration of a therapeutically effective amount of a compound of general formula (I)

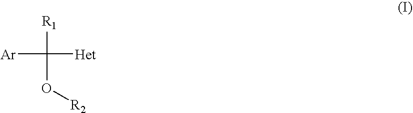

in which

Ar represents a phenyl radical or a thienyl radical, with no substitutions or optionally with 1, 2 or 3 equal or different substituent, selected from a group consisting of fluoride, chloride, bromide, methyl, trifluoromethyl and methoxy;

R₁ represents hydrogen or a lower alkyl group from C₁ to C₄;

R₂ represents a dialkyl(C₁-C₄)aminoalkyl (C₂-C₃), or azaheterocyclylalkyl, (C₂-C₃) radical; and

Het represents a five-armed nitrogenerated aromatic heterocycle that contains one to three nitrogen atoms, without substitutions or optionally substituted by 1 or 2 equal or different substituent selected from a group consisting of fluoride, chloride, bromide and methyl;

optionally in the form of its racemate, pure stereoisomers, especially enantiomers or diastereomers or in the form of mixtures of stereoisomers, especially enantiomers or diastereomers, in any suitable ratio;

in the form shown or in form of the acid or base or in form of a salt, especially a physiologically acceptable salt, or in form of a solvate, especially a hydrate.

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” is understood in particular, in the context of this invention, as salt formed either with a physiologically tolerated acid, that is to say salts of the particular active compound with inorganic or organic acids which are physiologically tolerated—especially if used on humans and/or mammals—or with at least one, preferably inorganic, cation which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydro-benzo[d]isothiazol-3-one (saccharin acid), monomethylsebacic acid, 5-oxo-proline, hexane-1-sulfonic acid, nicotinic acid, 2-, 3- or 4-aminobenzoic acid, 2,4,6-trimethyl-benzoic acid, alpha-lipoic acid, acetylglycine, acetylsalicylic acid, hippuric acid and/or aspartic acid. Examples of physiological tolerated salts of particular bases are salts of alkali metals and alkaline earth metals and with NH₄.

In the context of this invention the preferred salt is a salt of the particular active compound with a physiologically tolerated acid.

The salt particularly preferred in the context of this invention is the citrate.

In the context of this invention patient does mean any human being in need of treatment. In particular this encompasses man, woman and children. Depending on the specific kind of Renal colic encountered, the patient group most preferably treated can vary and at times (for example with stress incontinence) include more women, sometimes more elderly women, at times more men, especially elderly men, and sometimes, more children (for example in enuresis).

A preferred method according to the invention is characterized in that it comprises the administration of a compound of general formula (I), in which R₁ is selected from hydrogen or from a group consisting of methyl, ethyl, propyl, isopropyl, butyl, isobutyl, sec-butyl and tert-butyl.

Another preferred method according to the invention is characterized in that it comprises the administration of a compound of general formula (I), in which R₂ is selected from among a group consisting of dimethylaminoethyl, dimethylaminopropyl, diethylaminoethyl, piperidinylethyl, morpholinylpropyl and pirrolidinylethyl.

Another preferred method according to the invention is characterized in that it comprises the administration of a compound of general formula (Ia)

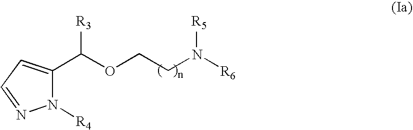

in which

n is 1 or 2;

R₃ is selected from:

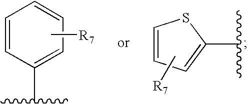

R₄ is selected from hydrogen, fluoride, chloride, bromide and methyl;

R₅ and R₆ are independently selected from lower C₍₁₋₄)-Alkyl or together with the Nitrogen form an azaheterocyclic ring;

R₇ is selected from the group consisting of hydrogen, fluoride, chloride, bromide, methyl, trifluoromethyl and methoxy.

A preferred method according to (this (above) aspect of) the invention is characterized in that it comprises the administration of a compound of general formula (Ia), in which R₇ is hydrogen.

Another preferred method according to (this (above) aspect of) the invention is characterized in that it comprises the administration of a compound of general formula (Ia), in which R₄ is Methyl.

Another preferred method according to (this (above) aspect of) the invention is characterized in that it comprises the administration of a compound of general formula (Ia), in which R₄ and its are either CH₃ or C₂H₅ or together with the Nitrogen form a piperidinyl, morpholinyl or pirrolidinyl ring.

Another preferred method according to (this (above) aspect of) the invention is characterized in that it comprises the administration of a compound of general formula (Ia) selected from among a group consisting of:

5-{α-[2-(dimethylamino)ethoxy]benzyl}-1H-pirazole,

(±)-5-{α-[2-(dimethylamino)ethoxy]benzyl}-1H-pirazole,

(+)-5-{α-[2-(dimethylamino)ethoxy]benzyl}-1H-pirazole,

(−)-5-{α-[2-(dimethylamino)ethoxy]benzyl}-1H-pirazole,

5-{α-[2-(dimethylamino)ethoxy]benzyl}-1H-pirazole citrate,

(±)-5-{α-[2-(dimethylamino)ethoxy]benzyl}-1H-pirazole citrate,

(+)-5-{α-[2-(dimethylamino)ethoxy]benzyl}-1H-pirazole citrate,

(−)-5-{α-[2-(dimethylamino)ethoxy]benzyl}-1H-pirazole citrate,

5-{α-[2-(dimethylamino)ethoxy]-2-thienylmethyl}-1-methyl-1H-pirazole,

(±)-5-{α-[2-(dimethylamino)ethoxy]-2-thienylmethyl}-1-methyl-1H-pirazole,

(+)-5-{α-[2-(dimethylamino)ethoxy]-2-thienylmethyl}-1-methyl-1H-pirazole,

(−)-5-{α-[2-(dimethylamino)ethoxy]-2-thienylmethyl}-1-methyl-1H-pirazole,

5-{α-[2-(dimethylamino)ethoxy]-2-thienylmethyl}-1-methyl-1H-pirazole citrate,

(±)-5-{α-[2-(dimethylamino)ethoxy]-2-thienylmethyl}-1-methyl-1H-pirazole citrate,

(+)-5-{α-[2-(dimethylamino)ethoxy]-2-thienylmethyl}-1-methyl-1H-pirazole citrate,

(−)-5-{α-[2-(dimethylamino)ethoxy]-2-thienylmethyl}-1-methyl-1H-pirazole citrate.

Another preferred method according to (this (above) aspect of) the invention is characterized in that it comprises the administration of a compound of general formula (Ib)

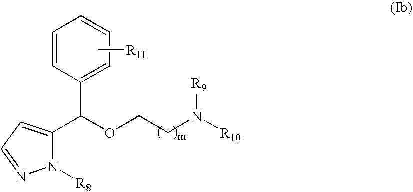

in which

m is 1 or 2;

R₈ a is selected from hydrogen, fluoride, chloride, bromide and methyl;

R₉ and R₁₀ are independently selected from lower C₍₁₋₄₎-Alkyl or together with the Nitrogen form an azaheterocyclic ring;

R₁₁ is selected from the group consisting of hydrogen, fluoride, chloride, bromide, methyl, trifluoromethyl and methoxy.

A preferred method according to (this (above) aspect of) the invention is characterized in that it comprises the administration of a compound of general formula (Ib), in which R₁₁ is hydrogen.

Another preferred method according to (this (above) aspect of ) the invention is characterized in that it comprises the administration of a compound of general formula (Ib), in which R₈ is Methyl.

Another preferred method to (this (above) aspect of) the invention is characterized in that it comprises the administration of a compound of general formula (Ib), in which R₈ and R₁₀ are either CH₈ or C₂H₅ or together with the Nitrogen form a piperidinyl, morpholinyl or pirrolidinyl ring;

preferably in which R₉ and R₁₀ are either CH₂ or C₂H₅;

especially in which R₉ and R₁₀ are equal and either CH₃ or C₂H₅;

most preferably in which R₉ and R₁₀ are both CH₃.

Another preferred method a according to (this (above) aspect of) the invention is characterized in that it comprises the administration of a compound of general formula (Ib), in which m is 1.

Another preferred method according to (this (above) aspect of) the invention is characterized in that it comprises the administration of a compound of general formula (Ib) selected from among a group consisting of:

5-{α-[2-(dimethylamino)ethoxy]benzyl}-1-methyl-1H-pirazole,

(±)-5-{α-[2-(dimethylamino)ethoxy]benzyl}-1-methyl-1H-pirazole

(+)-5-{α-[2-(dimethylamino)ethoxy]benzyl}-1-methyl-1H-pirazole

(−)-5-{α-[2-(dimethylamino)ethoxy]benzyl}-1-methyl-1H-pirazole

5-{α-[2-(dimethylamino)ethoxy]benzyl}-1-methyl-1H-pirazole citrate,

(±)-5-{α-[2-(dimethylamino)ethoxy]benzyl}-1-methyl-1H-pirazole citrate,

(+)-5-{α-[2-(dimethylamino)ethoxy]benzyl}-1-methyl-1H-pirazole citrate,

(−)-5-{α-[2-(dimethylamino)ethoxy]benzyl}-1-methyl-1H-pirazole citrate,

Another preferred method according to (this (above) aspect of) the invention is characterized in that it comprises the administration of

5-{α-[2-(dimethylamino)ethoxy]benzyl}-1-methyl-1H-pirazole,

Another preferred method according to (this (above) aspect of) the invention is characterized in that it comprises the administration of

(±)-5-{α-[2-(dimethylamino)ethoxy]benzyl}-1-methyl-1H-pirazole.

Another preferred method according to (this (above) aspect of) the invention is characterized in that it comprises the administration of

(+)-5-{α-[2-(dimethylamino)ethoxy]benzyl}-1-methyl-1H-pirazole.

Another preferred method according to (this (above) aspect of) the invention is characterized in that it comprises the administration of

(−)-5-{α-[2-(dimethylamino)ethoxy]benzyl}-1-methyl-1H-pirazole.

Another preferred method according to (this (above) aspect of) the invention is characterized in that it comprises the administration of

5-{α-[2-(dimethylamino)ethoxy]benzyl}-1-methyl-1H-pirazole citrate.

Another preferred method according to (this (above) aspect of) the invention is characterized in that it comprises the administration of

(±)-5-{α-[2-(dimethylamino)ethoxy]benzyl}-1-methyl-1H-pirazole citrate.

Another preferred method according to (this (above) aspect of) the invention is characterized in that it comprises the administration of

(+)-5-{α-[2-(dimethylamino)ethoxy]benzyl}-1-methyl-1H-pirazole citrate.

Another preferred method according to (this (above) aspect of) the invention is characterized in that it comprises the administration of

(−)-5-{α-[2-(dimethylamino)ethoxy]benzyl}-1-methyl-1H-pirazole citrate.

A preferred method according to the invention is characterized in that it comprises the administration of a compound of general formula (Ic)

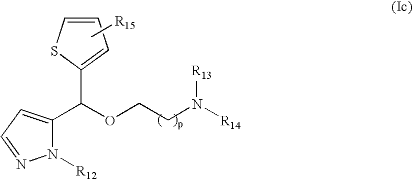

in which

p is 1 or 2;

R₁₂ is selected from hydrogen, fluoride, chloride, bromide and methyl;

R₁₃ and R₁₄ are independently selected from lower C₍₁₋₄₎-Alkyl or together with the Nitrogen form an azaheterocyclic ring;

R₁₅ is selected from the group consisting of hydrogen, fluoride, chloride, bromide, methyl, trifluoromethyl and methoxy.

A preferred method according to (this (above) aspect of) the invention is characterized in that it comprises the administration of a compound of general formula (Io). In which R₁₅ is hydrogen.

Another preferred method according to (this (above) aspect of) the invention is characterized in that it comprises the administration of a compound of general formula (Ic), in which R₁₂ is methyl.

Another preferred method according to (this (above) aspect of) the invention is characterized in that it comprises the administration of a compound of general formula (Ic), In which N₁₃ and R₁₄ are either CH₃ or C₂H₅ or together with the Nitrogen form a piperidinyl, morpholinyl or pirrolidinyl ring;

preferably in which R₁₃ and R₁₄ are either CH₃ or C₂H₅;

especially in which R₁₃ and R₁₄ are equal and either CH₃ or C₂H₅.

most preferably in which R₁ anti R₁₄ are both CH₃.

Another preferred method according to (this (above) aspect of) the invention is characterized in that it comprises the administration of a compound of general formula (Ic), in which p is 1.

Another preferred method according to (this (above) aspect of) the invention is characterized in that in comprises the administration of a compound of general formula (Ic) selected from among a group consisting of:

5-{α-[2-(dimethylamino)ethoxy]-2-thienylmethyl}-1-methyl-1H-pirazole,

(±)-5-{α-[2-(dimethylamino)ethoxy]-2-thienylmethyl}-1-methyl-1H-pirazole,

(+)-5-{α-[2-(dimethylamino)ethoxy]-2-thienylmethyl}-1-methyl-1H-pirazole,

(−)-5-{α-[2-(dimethylamino)ethoxy]-2-thienylmethyl}-1-methyl-1H-pirazole,

5-{α-[2-(dimethylamino)ethoxy]-2-thienylmethyl}-1-methyl-1H-pirazole citrate,

(±)-5-{α-[2-(dimethylamino)ethoxy]-2-thienylmethyl}-1-methyl-1H-pirazole citrate,

(+)-5-{α-[2-(dimethylamino)ethoxy]-2-thienylmethyl}-1-methyl-1H-pirazole citrate,

(−)-5-{α-[2-(dimethylamino)ethoxy]-2-thienylmethyl}-1-methyl-1H-pirazole citrate,

Another preferred method according to (this (above) aspect of) the invention is characterized in that it comprises the administration of:

5-{α-[2-(dimethylamino)ethoxy]-2-thienylmethyl}-1-methyl-1H-pirazole,

,optionally in the form of its racemate, pure stereoisomers, especially enantiomers or diastereomers or in the form of mixtures of stereoisomers, especially enantiomers or diastereomers, in any suitable ratio;

in form of the free base or in form of a salt, especially a physiologically acceptable salt, or in form of a solvate, especially a hydrate.

Another preferred method according to (this (above) aspect of) the invention is characterized in that it comprises the administration of:

5-{α-[2-(dimethylamino)ethoxy]-2-thienylmethyl}-1-methyl-1H-pirazole citrate,

,optionally in the form of is racemate, pure stereoisomers, especially enantiomers or diastereomers or in the form of mixtures of stereoisomers, especially enantiomers or diastereomers in any suitable ratio.

A preferred method according to the invention is characterized in that man means a female.

A preferred method according to the invention is characterized in that man means a male.

A preferred method according to this invention is characterized in that the patient is a woman.

A preferred method according to the invention is characterized in that the patient is an elderly woman.

A preferred method according to the invention is characterized in that the patient is a man.

A preferred method according to the invention is characterized in that the patient is an elderly man.

A preferred method according to the invention is characterized in that the patient is a child.

A preferred method according to the invention is characterized in that the therapeutically effective amount of the active compound is administered at a dose between 50 and 400 mg/day or between 200 and 600 mg/day.

In the context of this invention—if not clearly expressed as meaning the complete salt—dose means the dose of the active compound without the suit (which means without the counter ion, for example the citrate ion).

A preferred method according to the invention is characterized in that the compound is administered in form of a tablet or capsule.

A preferred method according to the invention is characterized in that the compound is administered in form of an immediate release formulation.

In the content of this invention “immediately release formulation” means any formulation with a release profile from which measured according to a standard measurement (e.g. using the paddle method according to the Pharmacopeia) (e.g. in 0.1% NaCl solution) within 30 minutes more than 60%, more preferably 60%, or even more preferably 70% of the active compound is released.

A speclally preferred aspeot is a method of treatnent of a patient suffering from renal colic characterized in that the method comprises the administration of a therapeutically effective amount of

5-{α-[2-(dimethylamino)ethoxy]benzyl}-1-methyl-1H-pirazole,

,optionally in the form of its racemate, pure stereoisomers, especially enantiomer or diastereomers or in the form of mixtures of stereoisomers, especially enantiomers or diastereomers, in any suitable ratio;

in form of the free base or in form of a salt, especially a physiologically acceptable salt, or in form of a solvate, especially a hydrate.

A preferred method according to the specially preferred aspect of the invention is characterized in that it comprises the administration of

5-{α-[2-(dimethylamino)ethoxy]benzyl}-1-methyl-1H-pirazole.

Another preferred method according to the specially preferred aspect of the invention is characterized in that it comprises the administration of

(+)-5-{α-[2-(dimethylamino)ethoxy]benzyl}-1-methyl-1H-pirazole,

Another preferred method according to the specially preferred aspect of the invention is characterized in that it comprises the administration of

(−)-5-{α-[2-(dimethylamino)ethoxy]benzyl}-1-methyl-1H-pirazole,

Another preferred method according to the specially preferred aspect of the invention is characterized in that it comprises the administration of

5-{α-[2-(dimethylamino)ethoxy]benzyl}-1-methyl-1H-pirazole citrate.

Another preferred method according to the specially preferred aspect of the invention is characterized in that it comprises the administration of

(±)-5-{α-[2-(dimethylamino)ethoxy]benzyl}-1-methyl-1H-pirazole citrate.

Another preferred method according to the specially preferred of the invention is characterized in that it comprises the administration of

(+)-5-{α-[2-(dimethylamino)ethoxy]benzyl}-1-methyl-1H-pirazole citrate.

Another preferred method according to the specially preferred aspect of the invention is characterized in that it comprises the administration of

(−)-5-{α-[2-(dimethylamino)ethoxy]benzyl}-1-methyl-1H-pirazole citrate.

Another preferred method according to the specially preferred aspect of the invention is characterized in that the patient is a woman.

Another preferred method according to the specially preferred aspect of the invention is characterized in that the patient is an elderly woman.

Another preferred method according to the specially preferred aspect of the invention is characterized in that the patient is a man.

Another preferred method according to the specially preferred aspect of the invention is characterized in that the patient is an elderly man.

Another preferred method according to the specially preferred aspect of the invention is characterized in that the patient is a child.

Another preferred method according to the specially preferred aspect of the invention is characterized in that the therapeutically effective amount of 5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that the therapeutically effective amount of (±)-5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that the therapeutically effective amount of (+)-5-{α-[2-(dimethylamino)ethoxy]benzyl}-1-methyl-1H-pirazole is administered at a dose between 50 and 400 mg/day or between 200 and 600 mg/day.

Another preferred method according to the specially preferred aspect of the invention is characterized in that the therapeutically effective amount of (−)-5-{α-[2-(dimethylamino)ethoxy]benzyl}-1-methyl-1H-pirazole, is administered at a dose between 50 and 400 mg/day or between 200 and 600 mg/day.

Another preferred method according to the specially preferred aspect of the invention is characterized in that 5-{α-[2-(dimethylamino)ethoxy]benzyl}-1-methyl-1H-pirazole is administered at a dose of 230 mg/day, 460 mg/day or 345 mg/day.

Another preferred method according to the specially preferred aspect of the invention is characterized in that (±)-5-{α-[2-(dimethylamino)ethoxy]benzyl}-1-methyl-1H-pirazole is administers at a dose of 280 mg/day. 345 mg/day, 460 mg/day or 575 mg/day, preferably 345 mg/day or 460 mg/day.

Another preferred method according to the specially preferred aspect of the invention is characterized in that 5-{α-[2-(dimethylamino)ethoxy]benzyl}-1-methyl-1H-pirazole citrate is administered at a dose of 400 mg/day, 600 mg/day, 800 mg/day or 1000 mg/day, preferably 600 mg/day or 800 mg/day.

Another preferred method according to the specially preferred aspect of the invention is characterized in that (±)-5-{α-[2-(dimethylamino)ethoxy]benzyl}-1-methyl-1H-pirazole, citrate is administered at a does of 400 mg/day, 600 mg/day, 800 mg/day or 1000 mg/day, preferably 600 mg/day of 800 mg/day.

Another preferred method according to the specially preferred aspect of the invention is characterized in that 5-{α-[2-(dimethylamino)ethoxy]benzyl}-1-methyl-1H-pirazole is administered twice daily.

Another preferred method according to the specially preferred aspect of the invention is characterized in that 5-{α-[2-(dimethylamino)ethoxy]benzyl}-1-methyl-1H-pirazole is administered orally.

Another preferred method according to the specially preferred aspect of the invention is characterized in that 5-{α-[2-(dimethylamino)ethoxy]benzyl}-1-methyl-1H-pirazole is administered in form of a tablet or capsule.

Another preferred method according to the specially preferred aspect of the invention is characterized in that 5-{α-[2-(dimethylamino)ethoxy]benzyl}-1-methyl-1H-pirazole is administered in form of an immediate release formulation.

Another preferred method according to the specially preferred aspect of the invention is characterized in that 5-{α-[2-(dimethylamino)ethoxy]benzyl}-1-methyl-1H-pirazole is administered in form of a formulation comprising any of the following:

sodium croscarmelose

colloidal silica dioxide,

a salt with stearic acid, especially magnesium stearate,

povidone,

microcrystalline cellulose

lactose monohydrate

polyethylene gycol.

Another preferred method according to the specially preferred aspect of the invention is characterized in that 5-{α-[2-(dimethylamino)ethoxy]benzyl}-1-methyl-1H-pirazole is administered in form of a formulation according to example 5.

Another preferred method according to the specially preferred aspect of the invention is characterized in that 5-{α-[2-(dimethylamino)ethoxy]benzyl}-1-methyl-1H-pirazole is administered in form of a formulation according to example 7.

Taking into amount its good pharmacodynamic properties, derivatives of aryl (or heteroaryl)azolylcarbinol, according to the invention, can be used satisfactorily in human and animal therapeutics to cure and relieve renal colic.

In human therapeutics, the dose administered of the compounds of the invention depends on the severity of the infection to be treated. It is normally between 50 and 400 mg/day or 200 and 600 mg/day. The compounds of the invention are administered for example in the form of capsules or tablets.

Any formulation or pharmaceutical composition according to the invention contains the active ingredient as well as optionally at least one auxiliary material and/or additive.

The auxiliary material and/or additive can be selected from carrier, excipient, support materials, glidants, filter, solvents, diluents, colorants, taste conditioners like sugar, antioxidants and/or binders. In the case of a suppository this might involve waxes or fatty acid esters or conserving agents, emulsifiers and/or carriers for parenteral application. The selection of these auxiliary materials and/or additives and of the amounts to be used depends upon how the pharmaceutical composition is to be applied.

Examples include here oral or parenteral like pulmonal, nasal, rectal and/or intravenous application. Therefore the pharmaceutical composition according to the invention can be adapted for topical or systemical application, especially dermal, subcutaneous, intramuscular, intra-articular and/or intraperitoneal, pulmonal, buccal, sublingual, nasal, percutanous, vaginal, oral or parenteral, pulmonal, nasal, rectal and/or intravenous application.

For oral application preparations in the form of tablets, chewable tablets, dagrees, capsules, granules, drops, juices and syrup are suitable. Solutions, suspensions, readily reconstitutable a dry preparations and sprays are suitable i.e. for parenteral application. The compounds according to the invention as a deposit in a dissolved form or in a patch, optionally with the addition of agents which promote dermal penetration, are examples of suitable percutaneous forms of application. Dermal applications include i.e. an ointment, a gel, a cream, a lotion, a suspension, an emulsion whereas the preferred form for rectal application is a suppository.

The examples and figures in the following section describing pharmacological trials are merely illustrative and the invention cannot be considered in any way as being restricted to these applications.

Figures:

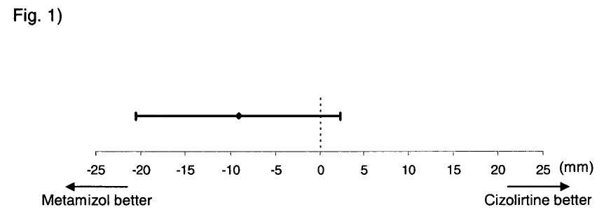

FIG. 1. Bilateral 95% confidence interval for the difference between the adjusted means of pain intensities as determined means of the VAS 30 minutes after the initiation of study drug administration (t+30 min)

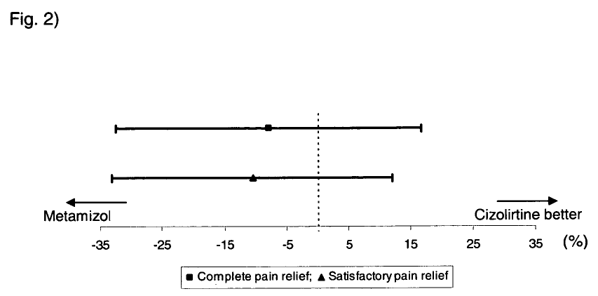

FIG. 2. Bilaterial 95% confidence intervals for the differonces between the proportions of patients showing complete and satisfactory pain relief as determined with the VAS at t+30 min

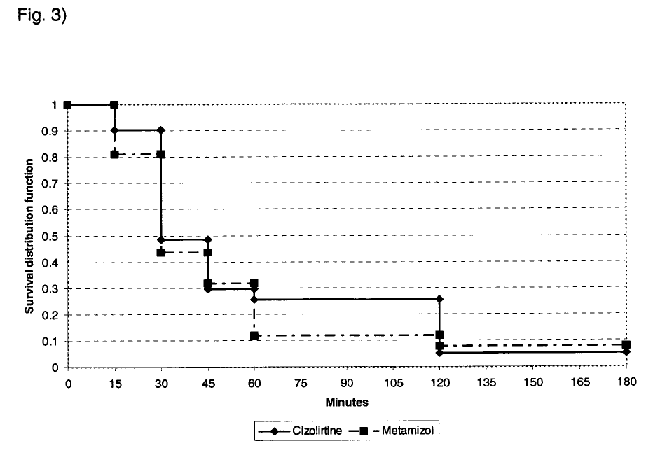

FIG. 3. Kaplan-Meier curve for time elapsed before complete relief of pain for the first three hours of the study

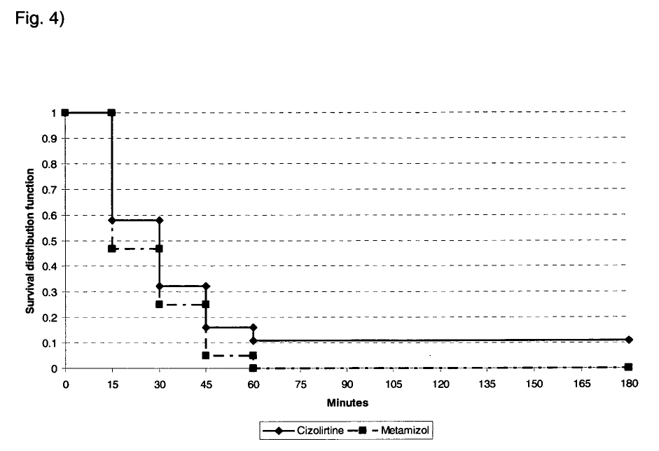

FIG. 4. Kaplan-Meier curve for time elapsed before satisfactory relief of pain for the first three hours of the study

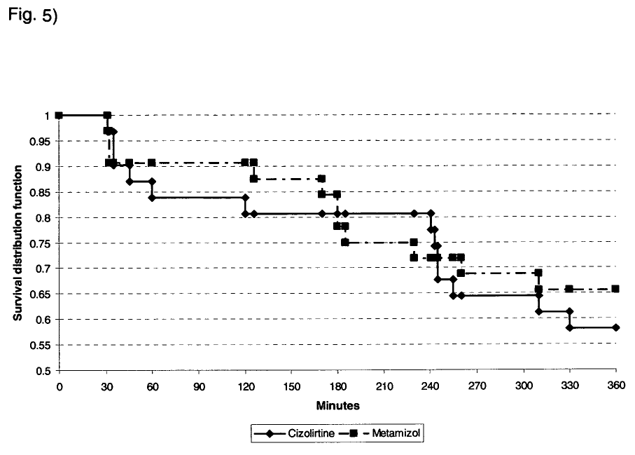

FIG. 5. Kaplan-Meier curve for time elapsed before the need for supplemental analgesia with butorphanol for the entire period of the study

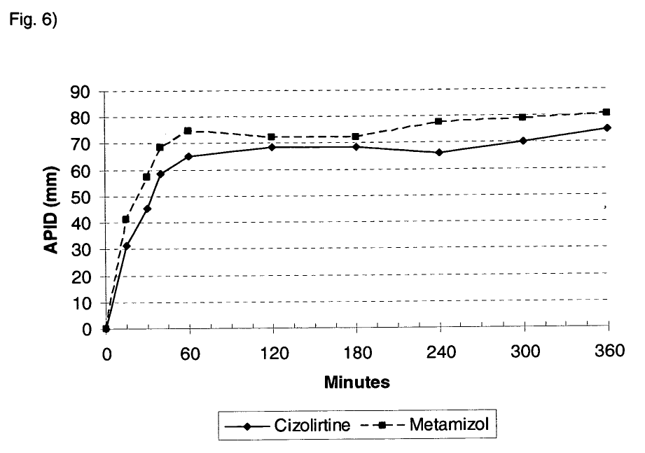

FIG. 6. Time-effect curve for APID for the entire period of the study

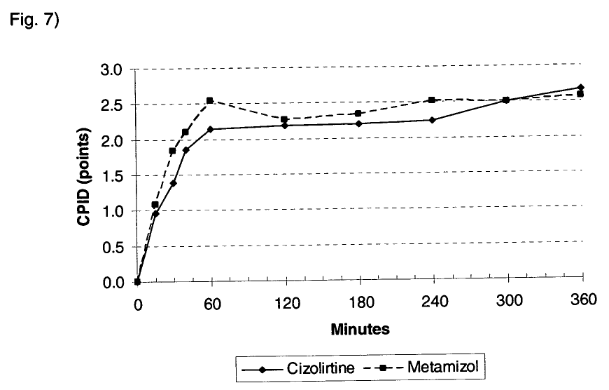

FIG. 7. Time-effect curve for CPID for the entire period of the study

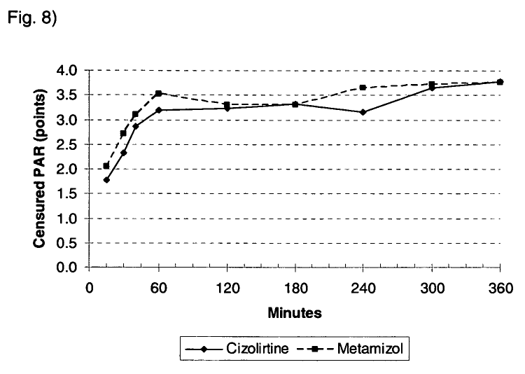

FIG. 8. Time-effect curve for PAR for the entire period of the study

### EXAMPLES

### Example 1

(±)-5-{α-[2-(dimethylamino)ethoxy]benzyl}-1-methyl-1H-pirazole or (±)-5-{α-[2-(dimethylamino)ethoxy]benzyl}-1-methyl-1H-pirazole citrate of the formula

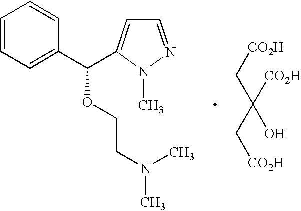

is already described in U.S. Pat. No. 6,017,596 and EP 289 380 B1 including its synthesis and analgetic properties. This compound is also known as cizolirtine.

### Example 1

Clinical Study A:

Abstract:

Objectives: to compare the analgesic efficacy and safety of cizolirtine citrate, an inhibitor of the release of the substance P, versus sodium metamizol to treat acute pain. Method: sixty four patients presenting with acute pain from suspected renal colic were randomized to a single intravenous dose of either 350 mg of cizolirtine citrate or 2,500 mg of sodium metamizol. Pain was assessed throughout 6 hours by means of visual analogical (VAS) and categorical scales. Results: renal colic was confirmed in all patients randomized. After 30 minutes, pain adjusted mean scores (100 mm VAS) were (LSM±SEM) 39.203±4.4 mm and 30.155±4.2 mm. In the cizolirtine and metamizol groups, respectively (p=0.1203). Although not significant, means differed by less than 10 mm, and the proportion of patients showing satisfactory pain relief (decrease of pain intensity ≧50%) at 30 minutes in the cizolirtine group was high (64.5%). Discussion: achieverment of analgesic activity by systemic blockade of substance P release is an interesting finding as blocking NK-1 receptors did not achieve analgesia in former studies. However, efficacy was lower than that of the comparator, an established treatment. Cizolirtine could be more effective to prevent the sensitization of dorsal spinal horn than as a plain analgesic.

Renal colic is one of the most common and excruciating pains, frequently caused by an acute partial ureteral obstruction due to a calculus. Pressure of the urine tends to increase causing an increase in tension of the walls of the ureter and renal pelvis which elicits pain. The urinary tract obstruction is a potent stimulus for renal prostaglandin synthesis, resulting in increased renal blood flow and diuresis. The emergency management of this condition aims to control the severe pain until the obstruction is relieved.

Non-steroidal anti-inflammatory drugs (NSAIDs) have been extensively evaluated to treat this condition, particularly in Europe. Most of the studies have shown comparable or better efficacy in comparison with opiolds as well as an advantageous safety profile1. However, some controversy remains at this point as prostaglandin synthetase inhibitors decrease diuresis by decreasing renal blood flow and glomerular filtration rate because of an increase in the pre-glomerular resistance. Besides, this might not be detected clinically because contralateral kidney function is not affected since NSAIDs would act selectively on the obstructed kidney and the serum creatinine may remain within normal limits.

The neurokinin substance P is produced and released mostly by primary nociceptive unmyelinated afferent fibres in their sensory endings causing a neurogenic inflammation as well as in their central terminals where it is involved in the generation of inflammation-evoked sensitization of spinal cord neurons. Cizolirtine citrate is a new drug with analgesic activity having the particularity of not belonging to either the NSAID or the opioid groups. Its mechanism of action is based in the inhibition of the release of substance P and CGRP in the spinal cord level2.

The purpose of this study was to compare the analgesic efficacy and safety of cizolirtine citrate versus sodium metamizol in patient presenting renal colic and to learn about the mechanisms through which substance P influences nociceptive processing.

Methods:

Study population: Subjects of either sex aged between 18 and 65 years old suffering moderate to severe pain due to a suspected renal colic starting within previous 24 hour and having a urine dipstick test positive for haematuria. Since this was an exploratory pilot phase II study, a formal sample size calculation was not performed. Moreover, no previous information regarding a comparison between the effects of sodium metamizol and placebo as analgesics for the management of renal colic could be found. Therefore, a minimum effect size to establish a non-inferiority margin was not be established. Nevertheless, to detect a difference of at least 25 mm in pain intensity measured with the VAS assuming a common standard deviation of 36.1 by means of a one-sided t test, 26 patients per group were necessary. Considering a dropout rate of 20%, 64 patients were recruited in the study

Study treatments: Either one single 350 mg dose of cizolirtine citrate or one single 2,500 mg dose of sodium metamizol by slow intravenous infusion in 15 minutes in a double-blind and randomized fashion and in a 1:1 proportion. Rescue doses of butorphanol were given as needed but were not allowed during the first 30 minutes.

Measurements: Patients ranked their pain intensity on both a visual analogical scale (VAS) and a verbal categorical scale at baseline and 15, 30 and 45 minutes and 1, 2, 3, 4, 6 and 6 hours after receiving the study drug. Vital signs were determined also at 15, 30 and 45 minutes and at 1 and 6 hours. Active questioning for adverse events since receiving study medication was done after 2 hours. Confirmatory diagnosis of an ureteral stone had to be established before the end of the study.

Primary endpoint: mean pain intensity as determined by the VAS 30 minutes after the start of study drug administration (t+30 min).

Statistics: Demographic and baseline continuous variables values were compared between treatment groups by means of a t test. Primary efficacy analyses were performed in the per protocol population. An analysis of covariance model with the factors treatment, centre and the treatment by centre interaction and considering the baseline value as a covariate was used or the comparison between the groups. In addition, the bilateral 95% confidence interval was calculated for the difference between treatments.

The proportions of patients with complete and satisfactory pain relief between treatments at t+30 min were compared between treatment groups by means of a chi-square test and also the bilateral 95% confidence intervals for the differences were calculated. The time-treatment variables were evaluated by means of the Kaplan-Meier method and the log-rank test was used to compare the treatment groups, pain intensity differences as determined by means of the visual analogical scale (APID), pain intensity differences as determined by means of the categorical scale (CPID) and relief of pain as determined by means of the categorical scale on pain relief (PAR) from baseline to t+30 min were compared between treatment groups by means of a repeated measures analysis of variance. For the corresponding cumulative variables a t test was used to compare SAPID, and a wilcoxon rank sum test for the SCPID and TOTPAR.

Results:

Sixty four patients (32 per treatment group) were randomized. All of them received the study drug and had postbaseline efficacy assessments available. Only one patient from the group of cizolirtine was excluded from the per protocol population (received a nephrostomy in addition o study medication to treat the colic).

Most of recruited patients were male. Physical examination findings, anthropometric parameters and mean pain intensity did not differ among groups at baseline. There were not statistically significant differences among groups for any of the parameters. However, differences in the pain intensity determined by the VAS were found among centres. Table 1 summarises the baseline characteristics of the study.

TABLE 1
Baseline characteristics of the patients in the per protocol population

| | | Cizolirtine (n = 31) | Metamizol (n = 32) | p-value |
|---|---|---|---|---|
| Gender: percentage of males | | 71.0 | 84.4 | — |
| Age (years: mean [SD]) | | 45.84 [12.29] | 42.63 [12.27] | 0.3031 |
| Weight (kg: mean [SD]) | | 80.94 [14.76] | 82.78 [12.48] | 0.5934 |
| Height (om: mean [SD]) | | 175 [10] | 179 [8] | 0.0994 |
| BMI (kg/m²: mean [SD]) | | 26.24 [3.8] | 25.71 [2.94] | 0.5316 |
| DBP (mm Hg: mean [SD]) | | 85.06 [11.73] | 82.38 [14.47] | 0.2714 |
| SBP (mm Hg: mean [SD]) | | 141.39 [15.31] | 141.16 [18.61] | 0.9574 |
| Heart rate (bpm: mean [SD]) | | 81.71 [11.23] | 79.5 [11.16] | 0.4364 |
| VAS pain score (mm: mean [SD]) all centres | | 79.42 [7.89] | 82.59 [10.5] | 0.1815 |
| VAS pain score (mm: mean [SD]) | Centre 1 | 80.81 [8.69] | 85.94 [7.38] | 0.0140 |
| | Centre 2 | 77.4 [10.2] | 70 [16.0] | (centre factor |
| | Cantres 4 + 5 + 6 | 78.2 [5.25] | 83.45 [7.78] | In the ANOVA) |

| | | | |
|---|---|---|---|
| Categorical pain score (mm: mean [SD]) | 2.94 [0.25] | 2.91 [0.3] | 0.6739 |

No supplemental doses of butorphanol were administered during the first 30 minutes of the study.

There were not significant differences between treatment groups in the primary efficacy endpoint (point estimate of the difference between adjusted means: 9.047 mm, see Table 2). The corresponding 95% confidence interval (from 2.35 to −20.489) is depicted in the FIG. 1. Value of this confidence interval would not permit to establish non-inferiority of cizolirtine respect to metamizol with any plausible non-inferiority margin. However, differences in pain reductions were a bit lower than the accepted threshold for clinical relevance (10 mm). The mean reductions in pain intensity determined by the VAS at t+30 min were 4.55 and 57.1 mm in the cizolirtine and metamizol groups, respectively; equivalent to a mean decrease of 57.2% and 69.1%, well under the threshold that definess satisfactory relief. These reductions were statistically significant within each of the groups. The proportions of patients at this time showing complete pain relief in the cizolirtine and metamizol groups respectively were 48.4% and 56.3%. Corresponding proportions for satisfacytory pain relief were 64.5% and 75.0%

The FIG. 2 shows the 95% confidence intervals for the differences betwen groups for both proportions. Althrough there was a slight trend favouring metamizol, the differences were not statistically significant with the sample size used in this

TABLE 2
Results of the primary efficacy endpoint (pain intensity at t + 30 min as
measured by means of the VAS) in the per protocol population.

| | | | Point | | |
|---|---|---|---|---|---|
| | Cizolirtine | Metamizol | estimate of | | |
| | (n = 31) | (n = 32) | the difference | LCI (mm) | UCI (mm) |
| Pain in the | 39.203 [4.400] | 30.155 | 9.047 | −20.489 | 2.395 |
| VAS at t + 30 min | | [4.203] | | | |
| (mm, LSM | | | | | |
| [SEM]) | | | | | |

Global p-value (ANCOVA): 0.1203

study.

Kaplan-Meier curves (FIGS. 3, 4 and 5) demonstrated that the proportion of patients showing complete and satisfactory pain relief over the course of the first 3 hours were not significantly different between the cizolirtine and metamizol group (p=0.5649 for differences in complete pain relief and 0.2071 for differences in satisfactory pain relief, log-rank test); as were not the proportion of patients needing supplemental analgesia with butorphanol after t+30 minutes (p=0.6045, log-rank test).

The time-curves for the derived endpoints APID, CPID and PAR censured by time of rescue medication are shown in the FIGS. 5 to 8. In general analgesia provided by cizolirtine was deferred with respect to that provided by metamizol, and this difference disappears after 2 hours. This delay would explain the trend favouring metamizol observed in pain intensities at t+30 min. The repeated measures analysis of variance over these endpoints from t=0 (start of study drug administration) to t+30 min did not reveal significant statistical differences in any of them. Results for SAPID, SCPID and TOTPAR are summarised on Table 3. There were not statistically significant differences. Corresponding non-censured endpoints showed similar results.

TABLE 3
Results of the derived endpoints SAPID, SCPID and TOTPAR for the
entire study period. Treatments were one single 350 mg intravenous
dose of cizolirtine citrate or one single 2.500 mg intravenous dose of
sodium metamizol

| | Cizolirtine | Metamizol | |
|---|---|---|---|
| | (n = 31) | (n = 32) | p-value |
| SAPID (mm, mean [SD]) | 398.24 [68.08] | 442.11 [78.46] | 0.2351 |
| SCPID (points, mean [SD]) | 13.39 [2.79] | 14.11 [3.48] | 0.2313 |
| TOTPAR (points, mean [SD]) | 19.67 [2.68] | 20.61 [2.67] | 0.1776 |

SAPID = sum of pain Intensity differences as determined by the visual analogical scale,
SCPID = sum of pain intensity differences as determined by means of a categorical scale on pain intensity,
TOTPAR = sum of pain relief scores as determined by means of a categorical scale on

Both treatments were very well tolerated. The number of adverse events was 5, 3 in the cizolirtine citrate group and 3 in the sodium metamizol group. These adverse events were vomits end one episode of fever. All were mild or moderate. None serious adverse event was reported.

Discussion and Conclusion:

This clinical trial was intended to assess the appropriateness and efficacy of cizolirtine administered by intravenous infusion as an analgesic agent in an acute pain model. Sodium metamizol was used as comparator in spite of the absence of historical data to establish the non-inferiority margin for testing the investigational drug. The results indicate that both drugs had an analgesic effect on renal colic.

In terms of proportion of patients achieving pain relief, both treatments showed good efficacy. Greater differences between treatments were present during the first hour, and almost disappeared beyond 2 hours. Most of the endpoints have evidenced differences as they focused the first 30 minutes and may be attributable to a slower action of the test drug rather than a lesser efficacy, which anyway is a disadvantage in the treatment of acute pain.

Higher doses could still improve efficacy, but the one employed here was close to the upper limit of the therapeutical range. The role of substance P as a sensitizer rather than a “pain transmitter” might explain the differential with the comparator and provides foundation for research in hyperalgesic states.

The clear limitation of this study is that in absence of a significant superiority of any of the treatments over the other the internal sensitivity can not be warranted: the absence of differences could be merely the lack of ability of the study to detect them although they exist. However, this was a pilot study that has shown analgesic efficacy with both treatments and provides information about the effect size that can be expected for futures reference in further clinical development of this drug.

### Example 3

### Example of Formulation for an Injectable (Im/Iv) Solution

| Citrate of (±)-5-{α-[2-(dimethylamino)ethoxy]benzyl}-1- | 50 | mg |
|---|---|---|
| methyl-1H-pirazole | | |
| 0.1 N Sodium hydroxide | o.s. pH 6 | |
| Water for injection o.s.p. | 1 | ml |

### Example 4

### Example of a Formulation (A) for a Tablet

| | |
|---|---|
| (±)-5-{α-[2-(dimethylamino)ethoxy]benzyl}-1-methyl- | 400 mg |
| 1H-pirazole citrate | |
| Sodium croscarmelose (Ac-DI-Sol) | 32 mg |
| Colloidal silica dioxide (Aerosyl 200) | 8 mg |
| Magnesium stearate, NF | 16 mg |
| Povidone K-30 | 40 mg |
| Microcrystalline cellulose (Avicel PH-102) | 146 mg |
| Lactose monohydrate (Farmatose 200M) | 158 mg |
| Total | 800 mg |

### Example 5

### Example of a Formulation (B) for a Tablet

| | |
|---|---|
| (±)-5-{α-[2-(dimethylamino)ethoxy]benzyl}-1-methyl- | 200 mg |
| 1H-pirazole citrate | |
| Sodium croscarmelose (Ac-Di-Sol) | 32 mg |
| Colloidal silica dioxide (Aerosyl 200) | 8 mg |
| Magnesium stearate, NF | 16 mg |
| Povidone K-30 | 40 mg |
| Microcrystalline cellulose (Avicel PH-102) | 246 mg |
| Lactose monohydrate (Farmatose 200M) | 258 mg |
| Total | 800 mg |

### Example 6

### Example of a Formulation (C) for a Tablet

| | |
|---|---|
| (±)-5-{α-[2-(dimethylamino)ethoxy]benzyl}-1-methyl- | 400 mg |
| 1H-pirazole citrate | |
| Sodium croscarmelose (Ac-Di-Sol) | 35 mg |
| Colloidal silica dioxide (Aerosyl 200) | 3 mg |
| Sodium steario fumarate | 12 mg |
| Polyethylene glycol 8000 | 30 mg |
| Microcrystalline cellulose (Avicel PH-102) | 75 mg |
| Lactose monohydrate (Farmatose 200M) | 45 mg |
| Total | 600 mg |

### Example 7

### Example of a Formulation of a Capsule

| | |
|---|---|
| (±)-5-{α-[2-(dimethylamino)ethoxy]benzyl}-1-methyl- | 200.0 mg |
| 1H-pirazole citrate | |
| Colloidal silica dioxide | 0.8 mg |
| Magnesium stearate | 2.4 mg |
| Lactose | 276.8 mg |
| Total | 480 mg |

1. A method of treating renal colic in a patient the method comprising administering to said patient a therapeutically effective amount of a carbinol compound of formula (II) 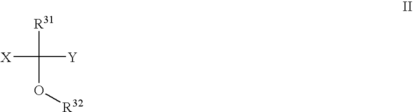
wherein
R³¹ represents a hydrogen atom, a linear or branched alkyl radical, a linear or branched alkenyl radical, an optionally at least mono-substituted cycloaliphatic radical optionally having at least one nitrogen atom as a ring member, or a phenyl radical,
R³² represents a hydrogen atom, a cycloaliphatic radical having a least one nitrogen atom as a ring member and optionally substituted with at least one radical independently selected from the group consisting of linear or branched alkyl radical optionally bound via a linear or branched alkylene group, —NR³³R³⁴, bound via linear or branched alkylene group, and —NR³⁵R³⁶ bound via a linear or branched alkylene group,
R³³ and R³⁴ independently represent a linear or branched alkyl radical or an unsubstituted benzyl radical,
R³⁵ and R³⁶ together with the nitrogen atom to which they are bonded represent a saturated, unsubstituted heterocyclic radical optionally having at least one further heteroatom as a ring member,
X represents a phenyl radical or a thienyl radical, wherein said phenyl radical or said thienyl radical is optionally substituted with at least one substituent independently selected from the group consisting of a linear or branched alkyl radical, a linear or branched alkoxy group, a linear or branched alkyl radical, which is at least partially halogenated and a halogen atom,
Y represents a heteroaryl radical, which contains one or more nitrogen atoms as ring members and which is unsubstituted or at least mono-substituted by one or more substituent independently from one another selected from the group consisting of a halogen atom, a linear or branched alkyl radical, a benzyl radical, a cyano group bound via a linear or branched C₁₋₄-alkylene group, a carboxy group bound via a linear or branched C₁₋₄-alkylene group, a methoxy carbonyl group bound via a linear or branched C₁₋₄-alkylene group, a hydroxy group bound via a linear or branched C₁₋₄-alkylene group, an amino group bound via a linear or branched C₁₋₄-alkylene group, a (C₁₋₄) dialkylamino group bound via a linear or branched C₁₋₄-alkylene group, and a cycloaliphatic radical, which contains at least one nitrogen atom as ring member and which is bound via a linear or branched C₁₋₄-alkylene group, or Y represents an unsubstituted heteroaryl radical, which contains two nitrogen atoms as ring members and which is condensed with a saturated, one methyl-substituted nitrogen atom as ring member containing cycloaliphatic group, or a stereoisomer, enantiomer diastereomer, or racemate thereof or a mixture of at least two stereoisomers enantiomers or diastereomers thereof or a corresponding salt or solvate thereof.

2-52. (canceled)

53. The method of claim 1, wherein R³¹ represents a hydrogen atom, a linear or branched C₁₋₄ alkyl radical, a linear or branched C₂₋₄ alkenyl radical, or a 5- or 6-membered cycloaliphatic radical, optionally having at least one nitrogen atom as a ring member and optionally substituted with at least one radical independently selected from the group consisting of linear or branched C₁₋₄ alkyl, phenyl, vinyl, cyclohexyl, and n-methyl-piperidyl.

54. The method of claim 1, wherein R³² represents a hydrogen atom, a 5- or 6-membered cycloaliphatic radical, optionally having at least one nitrogen atom as a ring member and optionally substituted with at least one radical independently selected from the group consisting of linear or branched C₁₋₄ alkyl, optionally bound via a linear or branched C₁₋₄-alkylene group, —NR³³R³⁴, bound via a linear or branched C₁₋₄ alkylene group, and —NR³⁵R³⁶, bound via a linear or branched C₁₋₄ alkylene group, or R³² represents a 5- or 6-membered cycloaliphatic radical, optionally having at least one nitrogen atom as a ring member and optionally substituted with at least one radical independently selected from the group consisting of linear or branched C₁₋₄-alkyl, optionally bound via a linear or branched C₁₋₄-alkylene group, —NR³³R³⁴, bound via a linear or branched C₂₋₃ alkylene group, and —NR³⁵R³⁶, bound via a linear or branched C₂₋₃ alkylene group.

55. The method of claim 1, wherein R³³ and R³⁴ independently represent a linear or branched C₁₋₄ alkyl radical or an unsubstituted benzyl radical.

56. The method of claim 1, wherein R³⁵ and R³⁶ together with the nitrogen atom to which they are bonded represent a saturated, unsubstituted 5- or 6-membered heterocyclic radical, optionally having at least one nitrogen atom as a ring member.

57. The method of claim 1, wherein X represents a phenyl radical or a thienyl radical, wherein said phenyl radical or said thienyl radical is optionally substituted with at least one substituent independently selected from the group consisting of a linear or branched C₁₋₄ alkyl radical, a linear or branched C₁₋₄ alkoxy radical, a linear or branched C₁₋₄ alkyl radical, which is at least partially fluorinated, a fluorine atom, a chlorine atom and a bromine atom.

58. The method of claim 1, wherein Y represents an azole radical selected from the group consisting of
a) a pyrazole of formula (a): 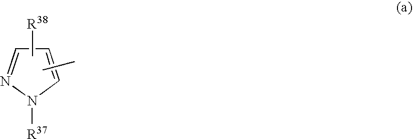
wherein R³⁷ represents a linear or branched C₁₋₁₂ alkyl radical, a benzyl radical or a radical of the formula: 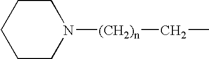
wherein n=1 or 2, and
R³⁸ represents a hydrogen atom, a methyl radical or a halogen atom,
b) an imidazole of the formula 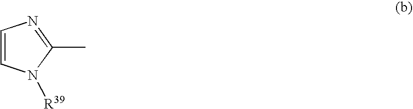
wherein R³⁹ represents a hydrogen atom, a C₁₋₁₂ alkyl radical, a benzyl radical, or a radical of the formula (b1):
R⁴⁰—(CH₂)ₙ— (b1)
wherein n is 2, 3 or 4 and R⁴⁰ represents a piperidinyl radical, a phenyl radical, a cyano group, a hydroxyl radical, a carboxy radical, an amino group, a dimethylamino group, or a methyl ester (CH₃—O—C(═O)—) group,
and
c) an imidazole of the formula:

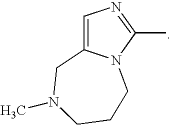

59. The method of claim 1, wherein
R³¹ represents a hydrogen atom, a methyl radical, an ethyl radical, an n-propyl radical, an iso-propyl radical, a sec-butyl radical, a tert-butyl radical, an n-butyl radical, a vinyl radical, a cyclohexyl radical, an N-methyl-piperidinyl group, or a phenyl group,
R³² represents a hydrogen atom, a dimethylaminoethyl group, a pyrrolidinylethyl group, a piperidinylethyl group, a methyl-benzyl-aminoethyl group, a morpholinylethyl group, a diisopropylaminoethyl group, a dimethylaminopropyl group, a piperidinylpropyl group, a pyrrolidinylpropyl group, a morpholinylpropyl group, an N-methyl-2-piperidyl group, an N-ethyl-2-piperidyl group, an N-propyl-2-piperidyl group, an N-methyl-2-pyrrolidinyl group, an N-ethyl-2-pyrrolidinyl group, an N-propyl-2-pyrrolidinyl group, or a 2-dimethylaminoethyl-1-methyl group,
X represents a phenyl radical, a 2-methyl-phenyl radical, a 3-methyl-phenyl radical, a 4-methyl phenyl radical, a 2-chloro-phenyl radical, a 3-chloro-phenyl radical, a 4-chloro-phenyl radical, a 2-fluoro-phenyl radical, a 3-fluoro-phenyl radical, a 4-fluoro-phenyl radical, a 2-trifluoromethyl-phenyl radical, a 3-trifluoromethyl-phenyl radical, a 4-trifluoromethyl-phenyl radical, a 2-methoxy-phenyl radical, a 3-methoxy-phenyl radical, a 4-methoxy-phenyl radical, a 3,4,5-tris-methoxy phenyl radical, a 3,4-dichloro-phenyl radical, a 2,4-dichloro-phenyl radical, a thien-2-yl radical, a thien-3-yl radical, a 3-methyl-thien-2-yl radical, a 5-methyl-thien-2-yl-radical, a 5-bromo-thien-2-yl radical or a 4-bromo-thien-2-yl-radical,
Y represents an azole radical selected from the group consisting of
a) a pyrazole of the formula (a): 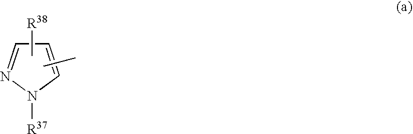
wherein
R³⁷ represents a methyl radical, an ethyl radical, an n-propyl radical, an iso-propyl radical, an n-butyl radical, a sec-butyl radical or a tert-butyl radical,
R³⁸ represents a hydrogen atom, a methyl radical, a bromine atom or a chlorine atom,
b) an imidazole of the formula 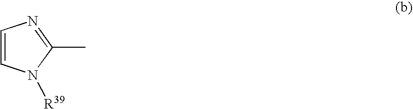
wherein R³⁹ represents a hydrogen atom, a methyl radical, an ethyl radical, an n-propyl radical, an iso-butyl radical, an n-butyl radical, a sec-butyl radical a tert-butyl radical, an n-pentyl radical, an n-hexyl radical, an n-heptyl radical, an n-octyl radical, an n-nonyl radical, an n-decyl radical, an n-undecyl radical an n-dodecyl radical, a benzyl radical, or a radical of the formula (b1):
R⁴⁰—(CH₂)ₙ—tm (b1)
wherein n is 2, 3 or 4 and R⁴⁰ represents a piperidinyl radical, a phenyl radical, a cyano group, a hydroxyl radical, a carboxy radical, an amino group, a dimethylamino group, or a methyl ester group,
c) an imidazole of the formula: 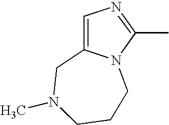
or a stereoisomer, enantiomer, diastereomer, or racemate thereof, or a mixture of at least two stereoisomers, enantiomers, or diastereomers thereof, or a corresponding salt or solvate thereof.

60. The method of claim 1, wherein said compound is selected from the group consisting of
[1] 2-{α-[2-(dimethylamino)ethoxy]benzyl}-1-methyl-1H-imidazole,
[2] 2-{4-chloro-α-[2-(dimethylamino)ethoxy]-methylbenzyl}-1-methyl-1H-imidazole,
[3] 2-{4-chloro-α-[2-(dimethylamino)ethoxy]benzyl}-1-methyl-1H-imidazole,
[4] 2-{3-chloro-α-[2-(dimethylamino)ethoxy]benzyl}-1-methyl-1H-imidazole,
[5] 2-{4-chloro-α-[2-(dimethylamino)ethoxy]-α-methylbenzyl}-1-methyl-1H-imidazole,
[6] 2-{4-fluoro-α-[2-(dimethylamino)ethoxy]-α-methylbenzyl}-1-methyl-1H-imidazole,
[7] 2-{α-[2-(dimethylamino)ethoxy]-α-methyl-3-(trifluoromethyl)benzyl}-1-methyl-1H-imidazole,
[8] 2-{3-chloro-α-[2-(dimethylamino)ethoxy]-α-methylbenzyl}-1-methyl-1H-imidazole,
[9] 2-{3-chloro-α-[2-(dimethylamino)ethoxy]-α-propylbenzyl}-1-methyl-1H-imidazole,
[10] 1-butyl-2-{4-chloro-α-[2-(dimethylamino)ethoxy]-α-methylbenzyl}-1H-imidazole,
[11] 2-{α-[2-(dimethylamino)ethoxy]-α-methyl-4-methoxybenzyl}-1-methyl-1H-imidazole,
[12] 2-{3-chloro-α-methyl-α-[2-(N-pyrrolidinyl)ethoxy]benzyl}-1-methyl-1H-imidazole,
[13] 2-{α-[2-(dimethylamino)ethoxy]-α-propyl-3,4,5-trimethoxybenzyl}-1-dodecyl-1H-imidazole,
[14] 1-butyl-2-{α-[2-(dimethylamino)ethoxy]-4-(trifluoromethyl)benzyl}-1H-imidazole,
[15] 1-methyl-2-{α-[2-methyl-α-[2-(N-piperidyl)ethoxy]-3-(trifluoromethyl)benzyl}-1H-imidazole,
[16] 2-{α-cyclohexyl-3,4-dichloro-α-[2-(dimethylamino)ethoxy]benzyl}-1-methyl-1H-imidazole,
[17] 2-{3,4-dichloro-α-[2-(dimethylamino)ethoxy]-α-propylbenzyl}-1-methyl-1H-imidazole,
[18] 2-{3,4-dichloro-α-[2-(dimethylamino)ethoxy]-α-methylbenzyl}-1-methyl-1H-imidazole,
[19] 2-{3,4-dichloro-α-[2-(dimethylamino)ethoxy]benzyl}-1-methyl-1H-imidazole,
[20] 2-{4-chloro-α-[2-(dimethylamino)ethoxy]-α-methylbenzyl}-1-[2-(N-piperidyl)ethyl]-1H-imidazole,
[21] 2-{4-chloro-α-[2-(dimethylamino)ethoxy]-α-methylbenzyl}-1-[2-(N-piperidyl)propyl]-1H-imidazole,
[22] 1-(3-cyanopropyl)-2-{4-chloro-α-[2-(dimethylamino)ethoxy]benzyl}-1H-imidazole,
[23] 2-{4-chloro-α-[2-(dimethylamino)ethoxy]-α-(N-methyl-4-piperidyl)benzyl}-1-methyl-1H-imidazole,
[24] 1-benzyl-2-{α-[(N-benzyl-N-methylamino)ethoxy]-4-chlorobenzyl}-1H-imidazole,
[25] 2-{4-chloro-α-[2-(dimethylamino)ethoxy]-α-methylbenzyl}-7-methyl-6,7,8,94-tetrahydro-1H-imidazole[1,5-a][1,4]diazepine,
[26] 2-{4-chloro-α-[2-(dimethylamino)ethoxy]benzyl}-7-methyl-6,7,8,9-tetrahydro-1H-imidazole[1,5-a][1,4]diazepine,
[27] 1-butyl-5-{α-[2-(dimethylamino)ethoxy]benzyl}-1H-pyrazole,
[28] 5-{α-(4-chlorophenyl)-α-[2-(dimethylamino)ethoxy]benzyl}-1-methyl-1H-pyrazole,
[29] 1-butyl-5-{α-[2-(dimethylamino)ethoxy]-3,4,5-trimethoxybenzyl}-1H-pyrazole
[30] 1-butyl-5-{4-chloro-α-[2-(dimethylamino)ethoxy]-α-methylbenzyl}-1H-pyrazole,
[31] 5-{α-[2-(dimethylamino)ethoxy]benzyl}-1-methyl-1H-pyrazole,
[32] 5-{α-[2-(dimethylamino)ethoxy]-α-methylbenzyl}-1-methyl-1H-pyrazole,
[33] 5-{α-[2-(dimethylamino)ethoxy]-3,4,5-trimethoxybenzyl}-1-methyl-1H-pyrazole,
[34] 1-methyl-5-{α-[2-(N-pyrrolidinyl)ethoxy]benzyl}-1H-pyrazole,
[35] 1-methyl-5-{α-[2-(N-morpholinyl)ethoxy]benzyl}-1H-pyrazole,
[36] 5-{α-[2-(dimethylamino)ethoxy]-α-methyl-3,4,5-trimethoxybenzyl}-1-methyl-1H-pyrazole,
[37] 4-bromo-5-{α-[2-(dimethylamino)ethoxy]benzyl}-1-methyl-1H-pyrazole,
[38] 1,3-dimethyl-5-{α-[2-(dimethylamino)ethoxy]-α-methylbenzyl}-1H-pyrazole,
[39] 1,3-dimethyl-5-{α-[2-(dimethylamino)ethoxy]benzyl}-1H-pyrazole,
[40] 5-{α-[2-(dimethylamino)ethoxy]-2-methylbenzyl}-1-methyl-1H-pyrazole,
[41] 4-chloro-5-{4-chloro-α-[2-(dimethylamino)ethoxy]benzyl}-1-methyl-1H-pyrazole,
[42] 5-{4-chloro-α-[2-(dimethylamino)ethoxy]benzyl}-1-methyl-1H-pyrazole,
[43] 5-{3-chloro-α-[2-(dimethylamino)ethoxy]benzyl}-1-methyl-1H-pyrazole,
[44] 5-{α-[2-(dimethylamino)ethoxy]-4-methylbenzyl}-1-methyl-1H-pyrazole,
[45] 5-{2-chloro-α-[2-(dimethylamino)ethoxy]benzyl}-1-methyl-1H-pyrazole,
[46] 1-methyl-5-{α-[2-(N-piperidyl)ethoxy]benzyl}-1H-pyrazole,
[47] 1-methyl-5-{α-[2-(N-propyl-2-piperidyl)ethoxy]benzyl}-1H-pyrazole,
[48] 5-{α-[2-(N-ethyl-2-piperidyl)ethoxy]benzyl}-1-methyl-1H-pyrazole,
[49] 1-methyl-5-{α-[2-(N-methyl-2-pyrrolidinyl)ethoxy]benzyl}-1H-pyrazole,
[50] 5-{α-[2-(diisopropylamine)ethoxy]benzyl}-1-methyl-1H-pyrazole,
[51] 1-methyl-5-{α-[2-(N-methyl-2-piperidyl)ethoxy]benzyl}-1H-pyrazole,
[52] 2-{4-chloro-α-[3-(dimethylamino)propoxy]-α-methylbenzyl}-1-methyl-1H-imidazole,
[53] 2-{3-chloro-α-[3-(dimethylamino)propoxy]benzyl}-1-methyl-1H-imidazole,
[54] 2-{4-chloro-α-[3-(dimethylamino)propoxy]-α-ethylbenzyl}-1-methyl-1H-imidazole,
[55] 2-{α-butyl-3-chloro-α-[3-(dimethylamino)propoxy]benzyl}-1-methyl-1H-imidazole,
[56] 2-{α-cyclohexyl-4-chloro-α-[3-(dimethylamino)propoxy]benzyl}-1-methyl-1H-imidazole,
[57] 2-{α-[3-(dimethylamino)propoxy]-4-fluoro-α-methylbenzyl}-1-methyl-1H-imidazole,
[58] 2-{α-[3-(dimethylamino)propoxy]-α-methyl-3-(trifluoromethyl)benzyl}-1-methyl-1H-imidazole,
[59] 2-{2-chloro-α-[3-(dimethylamino)propoxy]-α-methylbenzyl}-1-methyl-1H-imidazole,
[60] 2-{3-chloro-α-[3-(dimethylamino)propoxy]-α-methylbenzyl}-1-methyl-1H-imidazole,
[61] 2-{α-[3-(dimethylamino)propoxy]-α-methyl-3,4,5-trimethoxybenzyl}-1-methyl-1H-imidazole,
[62] 2-{α-[3-(dimethylamino)propoxy]-α-methyl-4-methoxybenzyl}-1-methyl-1H-imidazole,
[63] 2-{4-chloro-α-[3-(dimethylamino)propoxy]benzyl}-1-methyl-1H-imidazole,
[64] 2-{α-[3-(dimethylamino)propoxy]-3,4,5-trimethoxybenzyl}-1-methyl-1H-imidazole,
[65] 2-{α-[3-(dimethylamino)propoxy]-α-methyl-4-(trifluoromethyl)benzyl}-1-methyl-1H-imidazole,
[66] 2-{α-[3-(dimethylamino)propoxy]-3-(trifluoromethyl)benzyl}-1-methyl-1H-imidazole,
[67] 2-{α-[3-(dimethylamino)propoxy]-4-(trifluoromethyl)benzyl}-1-methyl-1H-imidazole,
[68] 2-{α-[3-(dimethylamino)propoxy]-4-methoxybenzyl}-1-methyl-1H-imidazole,
[69] 2-{α-butyl-α-[3-(dimethylamino)propoxy]-3-(trifluoromethyl)benzyl}-1-methyl-1H-imidazole,
[70] 1-butyl-2-{4-chloro-α-[3-(dimethylamino)propoxy]-α-methylbenzyl}-1H-imidazole,
[71] 1-butyl-2-{α-butyl-α-[3-(dimethylamino)propoxy]-3,4,5-trimethoxybenzyl}-1H-imidazole,
[72] 1-butyl-2-{α-butyl-2-chloro-α-[3-(dimethylamino)propoxy]benzyl}-1H-imidazole
[73] 1-butyl-2-{α-butyl-2,4-dichloro-α-[3-(dimethylamino)propoxy]benzyl}-1H-imidazole,
[74] 1-butyl-2-{α-[3-(dimethylamino)propoxy]-4-(trifluoromethyl)benzyl}-1H-imidazole,
[75] 2-{4-chloro-α-[3-(N-piperidyl)propoxy]benzyl}-1-methyl-1H-imidazole,
[76] 1-methyl-2-{α-methyl-α-[3-(N-piperidyl)propoxy]-4-trifluoromethyl)benzyl}-1H-imidazole,
[77] 2-{α-butyl-2-chloro-α-[3-(dimethylamino)propoxy]benzyl}-1-methyl-1H-imidazole,
[78] 2-{α-butyl-3,4-dichloro-α-[3-(dimethylamino)propoxy]benzyl}-1-methyl-1H-imidazole,
[79] 2-{3,4-dichloro-α-[3-(dimethylamino)propoxy]-α-methylbenzyl}-1-methyl-1H-imidazole,
[80] 2-{3,4-dichloro-α-[3-(dimethylamino)propoxy]benzyl}-1-methyl-1H-imidazole,
[81] 2-{α-cyclohexyl-3,4-dichloro-α-[3-(dimethylamino)propoxy]benzyl}-1-methyl-1H-imidazole,
[82] 2-{4-chloro-α-[3-(dimethylamino)propoxy]-α-methylbenzyl}-α-[2-(N-piperidyl)ethyl]-1H-imidazole,
[83] 2-{4-chloro-α-[3-(dimethylamino)propoxy]-α-methylbenzyl}-1-[2-(N-piperidyl)propyl]-1H-imidazole,
[84] 2-{4-chloro-α-[3-(dimethylamino)propoxy]-α-(N-methyl-4-piperidyl)benzyl}-1-methyl-1H-imidazole,
[85] 1-butyl-5-{α-[3-(dimethylamino)propoxy]benzyl}-1H-imidazole,
[86] 1-butyl-5-{4-chloro-α-[3-(dimethylamino)propoxy]-α-methylbenzyl}-1H-pyrazole,
[87] 5-{α-[3-(dimethylamino)propoxy]benzyl}-1-methyl-1H-pyrazole,
[88] 5-{α-[3-(dimethylamino)propoxy]-α-methylbenzyl}-1-methyl-1H-pyrazole,
[89] 1,3-dimethyl-5-{α-[3-(dimethylamino)propoxy]-α-methylbenzyl}-1H-pyrazole,
[90] 1,3-dimethyl-5-{α-[3-(dimethylamino)propoxy]benzyl}-1H-pyrazole,
[91] 5-{α-[3-(dimethylamino)propoxy]-2-methylbenzyl}-methyl-1H-pyrazole,
[92] 5-chloro-5-{4-chloro-α-[3-(dimethylamino)propoxy]benzyl}-1-methyl-1H-pyrazole,
[93] 1-methyl-5-{α-[3-(dimethylamino)propoxy]benzyl}-1H-pyrazole,
[94] 1-methyl-5-{α-[3-(dimethylamino)propoxy]benzyl}-1H-pyrazole,
[95] 4-{4-chloro-α-[2-(dimethylamino)ethoxy]benzyl}-1-methyl-1H-pyrazole,
[96] 4-{4-chloro-α-[2-(dimethylamino)ethoxy]-α-methylbenzyl}-1-methyl-1H-pyrazole,
[97] 4-{4-chloro-α-[2-(N-propyl-2-piperidyl)ethoxy]benzyl}-1-methyl-1H-pyrazole,
[98] 4-{4-chloro-α-[2-(N-methyl-2-piperidyl)ethoxy]benzyl}-1-methyl-1H-pyrazole,
[99] 4-{4-chloro-α-[2-(N-ethyl-2-piperidyl)ethoxy]benzyl}-1-methyl-1H-pyrazole,
[100] 4-{4-chloro-α-[2-(diisopropylamino)ethoxy]benzyl}-1-methyl-1H-pyrazole,
[101] 4-{4-chloro-α-[2-(N-methyl-2-pyridinyl)ethoxy]benzyl}-1-methyl-1H-pyrazole,
[102] 4-{α-[3-(dimethylamino)propoxy]benzyl}-1-methyl-1H-pyrazole,
[103] 4-{4-chloro-α-[3-(N-morpholinyl)propoxy]benzyl}-1-methyl-1H-pyrazole,
[104] 4-{4-chloro-α-[3-(N-pyrrolidinyl)propoxy]benzyl}-1-methyl-1H-pyrazole,
[105] 2-(α-hydroxybenzyl)-1H-imidazole,
[106] 2-(4-chloro-α-hydroxybenzyl)-1H-imidazole,
[107] 2-(4-chloro-α-hydroxybenzyl)-1-methyl-1H-imidazole,
[108] 2-(3-chloro-α-hydroxybenzyl)-1-methyl-1H-imidazole,
[109] 2-(4-fluoro-α-hydroxybenzyl)-1-methyl-1H-imidazole,
[110] 2-[α-hydroxy-3-(trifluoromethyl)benzyl]-1-methyl-1H-imidazole,
[111] 2-[α-hydroxy-4-(trifluoromethyl)benzyl]-1-methyl-1H-imidazole,
[112] 2-(α-hydroxy-3,4,5-trimethoxybenzyl)-1-methyl-1H-imidazole,
[113] 2-(3,4-dichloro-α-hydroxybenzyl)-1-methyl-1H-imidazole,
[114] 1-butyl-2-[α-hydroxy-4-(trifluoromethyl)benzyl]-1H-imidazole,
[115] 1-butyl-2-(3,4-dichloro-α-hydroxybenzyl)-1H-imidazole,
[116] 1-butyl-2-(4-chloro-α-hydroxybenzyl)-1H-imidazole,
[117] 1-butyl-2-(α-hydroxy-3,4,5-trimethoxybenzyl)-1H-imidazole,
[118] 1-dodecyl-2-(α-hydroxy-3,4,5trimethoxybenzyl)-1H-imidazole,
[119] 2-(α-butyl-3-chloro-α-hydroxybenzyl)-1-methyl-1H-imidazole,
[120] 2-(3-chloro-α-hydroxy-α-methylbenzyl)-1-methyl-1H-imidazole,
[121] 2-(4-chloro-α-hydroxy-α-methylbenzyl)-1-methyl-1H-imidazole,
[122] 2-[4-chloro-α-hydroxy-α-(N-methyl-4-piperidyl)benzyl]-1-methyl-1H-imidazole,
[123] 2-(4-chloro-α-ethyl-α-hydroxybenzyl)-1-methyl-1H-imidazole,
[124] 2-(α-butyl-4-chloro-α-hydroxybenzyl)-1-methyl-1H-imidazole,
[125] 2-(α-cyclohexyl-4-chloro-α-hydroxybenzyl)-1-methyl-1H-imidazole,
[126] 2-(2-chloro-α-hydroxy-α-methylbenzyl)-1-methyl-1H-imidazole,
[127] 2-(α-butyl-2-chloro-α-hydroxybenzyl)-1-methyl-1H-imidazole,
[128] 2-[α-hydroxy-α-methyl-3-(trifluoromethyl)benzyl]-1-methyl-1H-imidazole,
[129] 2-[α-butyl-α-hydroxy-3-(trifluoromethyl)benzyl]-1-methyl-1H-imidazole,
[130] 2-[α-cyclohexyl-α-hydroxy-3-(trifluoromethyl)benzyl]-1-methyl-1H-imidazole,
[131] 2-[α-hydroxy-α-methyl-4-(trifluoromethyl)benzyl]-1-methyl-1H-imidazole,
[132] 2-(4-fluoro-α-hydroxy-α-methylbenzyl)-1H-imidazole,
[133] 2-(α-hydroxy-α-methyl-4-methoxybenzyl)-1-methyl-1H-imidazole,
[134] 2-(3,4-dichloro-α-hydroxy-α-methylbenzyl)-1-methyl-1H-imidazole,
[135] 2-(α-butyl-3,4-dichloro-α-hydroxybenzyl)-1-methyl-1H-imidazole,
[136] 2-(α-cyclohexyl-3,4-dichloro-α-hydroxybenzyl)-1-methyl-1H-imidazole,
[137] 2-(α-hydroxy-α-methyl-3,4,6-trimethoxybenzyl)-1-methyl-1H-imidazole,
[138] 1-butyl-2-(4-chloro-α-hydroxy-α-methylbenzyl)-1H-imidazole,
[139] 1-butyl-2-(α-butyl-4-chloro-α-hydroxybenzyl)-1H-imidazole,
[140] 1-butyl-2-[4-chloro-α-hydroxy-α-(N-methyl-4-piperidyl)benzyl]-1H-imidazole,
[141] 1-butyl-2-(α-butyl-α-hydroxy-3,4,5-trimethoxybenzyl)-1H-imidazole,
[142] 1-butyl-2-(α-butyl-2-chloro-α-hydroxybenzyl)-1H-imidazole,
[143] 1-butyl-2-[α-ethyl-α-hydroxy-3-(trifluoromethyl)benzyl]-1H-imidazole,
[144] 1-butyl-2-(α-butyl-2,4-dichloro-α-hydroxybenzyl)-1H-imidazole,
[145] 2-(4-chloro-α-hydroxy-α-methylbenzyl)-1-[2-(N-piperidyl)ethyl]-1H-imidazole,
[146] 2-(4-chloro-α-hydroxy-α-methylbenzyl)-1-(3-dimethylaminopropyl)-1H-imidazole,
[147] 2-(α-butyl-α-hydroxy-3,4,5-trimethoxybenzyl)-1-dodecyl-1H-imidazole,
[148] 1-benzyl-2-[α-butyl-α-hydroxy-3-(trifluoromethyl)benzyl]-1H-imidazole,
[149] 1-benzyl-2-(4-chloro-α-hydroxy-α-methylbenzyl)-1H-imidazole,
[150] 1-(2-cyanoethyl)-2-(4-chloro-α-hydroxybenzyl)-1H-imidazole
[151] 1-(3-aminopropyl)-2-(4-chloro-α-hydroxybenzyl)-1H-imidazole,
[152] 3-[2-(3-chloro-α-hydroxybenzyl)-1H-imidazole-1-yl]propanoic aid,
[153] 2-(4-chloro-α-hydroxybenzyl)-1-(3-hydroxypropyl)-1H-imidazole,
[154] 3-[2-(3-chloro-α-hydroxybenzyl)-1H-imidazole-1-yl]methyl-propanoate,
[155] 2-(α-hydroxybenzyl)-1-(3hydroxypropyl)-1H-imidazole,
[156] 2-(α-hydroxy-4-methylbenzyl)-1-(3-hydroxypropyl)-1H-imidazole,
[157] 2-(α-hydroxy-4-methoxylbenzyl)-1-(3-hydroxypropyl)-1H-imidazole,
[158] 2-(3,4-dichloro-α-hydroxybenzyl)-1-(3-hydroxypropyl)-1H-imidazole,
[159] 3-{2-(α-hydroxybenzyl)-1H-imidazole-yll}-methyl propanoate,
[160] 2-(4-chloro-α-hydroxybenzyl)-1-(4-hydroxybutyl)-1H-imidazole,
[161] 1-(3-cyanopropyl)-2-(4-chloro-α-hydroxybenzyl)-1H-imidazole,
[162] 4-[2-(4-chloro-α-hydroxybenzyl)-1H-imidazole-1-yl]butanoic acid.
[163] 4-[2-(4-chloro-α-hydroxybenzyl)-1H-imidazole-1-yl]-methyl butanoate.
[164] 1-butyl-5-(α-hydroxybenzyl)-1H-pyrazole,
[165] 5-(4-chloro-α-hydroxybenzyl)-1-methyl-1H-pyrazole,
[166] 5-(α-hydroxy-3,4,5-trimethoxybenzyl)-1-methyl-1H-pyrazole,
[167] 1-butyl-5-(α-hydroxy-3,4,5-trimethoxybenzyl)-1H-pyrazole,
[168] 4-bromo-5-(α-hydroxybenzyl)-1-methyl-1H-pyrazole,
[169] 5-[α-(4-chlorophenyl)-α-hydroxybenzyl]-1-methyl-1H-pyrazole,
[170] 1-butyl-5-(4-chloro-α-hydroxy-α-methylbenzyl)-1H-pyrazole,
[171] 5-(α-hydroxy-α-methylbenzyl)-1-methyl-1H-pyrazole,
[172] 5-(α-hydroxy-α-methyl-3,4,5-trimethoxybenzyl)-1-methyl-1H-pyrazole,
[173] 1,3-dimethyl-5-(α-hydroxy-α-methylbenzyl)-1H-pyrazole,
[174] 1-butyl-5-(α-hydroxy-α-vinylbenzyl)-1H-pyrazole,
[175] 1-butyl-5-(4-chloro-α-hydroxy-α-vinylbenzyl)-1H-pyrazole,
[176] 4-chloro-5-(α-hydroxybenzyl)-1-methyl-1H-pyrazole,
[177] 5-(α-hydroxy-2-methylbenzyl)-1-methyl-1H-pyrazole,
[178] 5-(3-chloro-α-hydroxybenzyl)-1-methyl-1H-pyrazole,
[179] 5-(α-hydroxy-4-methylbenzyl)-1-methyl-1H-pyrazole,
[180] 5-(2-chloro-α-hydroxybenzyl)-1-methyl-1H-pyrazole,
[181] 5-(α-hydroxy-4-methoxybenzyl)-1-methyl-1H-pyrazole,
[182] 5-{α-[2-(dimethylamino)ethoxy]-2-thienylmethyl}-1-methyl-1H-pyrazole,
[183] 5-{α-[2-(dimethylamino)ethoxy]-2-thienylmethyl}-1-methyl-1H-pyrazole citrate,
[184] 5-{α-[2-(dimethylamino)ethoxy]-3-thienylmethyl}-1-methyl-1H-pyrazole,
[185] 2-{α-[2-(dimethylamino)ethoxy]-2-thienylmethyl}-1-methyl-1H-imidazole,
[186] 5-{α-[2-(dimethylamino)ethoxy]-3-methyl-2-thienylmethyl}-1-methyl-1H-pyrazole,
[187] 5-{α-[2-(dimethylamino)ethoxy]-5-methyl-2-thienylmethyl}-1-methyl-1H-pyrazole,
[188] 5-{5-bromo-α-[2-(dimethylamino)ethoxy]-2-thienylmethyl}-1-methyl-1H-pyrazole,
[189] 5-{4-bromo-α-[2-(dimethylamino)ethoxy]-2-thienylmethyl}-1-methyl-1H-pyrazole,
[190] 5-{α-[2-(dimethylamino)ethoxy]-α-methyl-2-thienylmethyl}-1-methyl-1H-pyrazole,
[191] 5-{α-[2-(dimethylamino)ethoxy]benzyl}-1-methyl-1H-pyrazole citrate,
[192] (±)-5-{α-[2-(dimethylamino)-1-(methyl)ethoxy]benzyl}-1-methyl-1H-pyrazole,
[193] (±)-5-{α-[2-(dimethylamino)-1-(methyl)ethoxy]benzyl}-1-methyl-1H-pyrazole,
[194] (+)-5-{α-[2-(dimethylamino)ethoxy]-2-thienylmethyl}-1-methyl-1H-pyrazole,
[195] (−)-5-{α-[2-(dimethylamino)ethoxy]-2-thienylmethyl}-1-methyl-1H-pyrazole,
[196] (+)-5-{α-[2-(dimethylamino)ethoxy]-2-thienylmethyl}-1-methyl-1H-pyrazole citrate,
[197] (−)-5-{α-[2-(dimethylamino)ethoxy]-2-thienylmethyl}-1-methyl-1H-pyrazole citrate,
[198] (+)-5-{α-[2-(dimethylamino)ethoxy]-2-thienylmethyl}-1-methyl-1H-pyrazole-D-ditoluyitartrat,
[199] (−)-5-{α-[2-(dimethylamino)ethoxy]-2-thienylmethyl}-1-methyl-1H-pyrazole D-ditoluyitartrat,
[200] (+)-5-{α-[2-(dimethylamino)ethoxy]benzyl}-1-methyl-1H-pyrazole citrate,
[201] (−)-5-{α-[2-(dimethylamino)ethoxy]benzyl}-1-methyl-1H-pyrazole citrate,
[202] 5-(α-hydroxy-2-thienylmethyl)-1-methyl-1H-pyrazole,
[203] 5-(α-hydroxy-3-methyl-2-thienylmethyl)-1-methyl-1H-pyrazole,
[204] 5-(α-hydroxy-5-methyl-2-thienylmethyl)-1-methyl-1H-pyrazole,
[205] 5-(5-bromo-α-hydroxy-2-thienylmethyl)-1-methyl-1H-pyrazole,
[206] 5-(4-bromo-α-hydroxy-2-thienylmethyl)-1-methyl-1H-pyrazole and
[207] 5-(α-hydroxy-α-methyl-2-thienylmethyl)-1-methyl-1H-pyrazole.

61. A method of treating renal colic in a patient, the method comprising administering to said patient a therapeutically effective amount of a compound of formula (I) 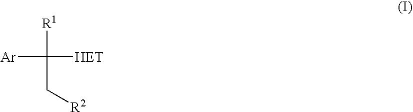
wherein
Ar represents a phenyl radical or a thienyl radical, wherein said phenyl radical or said thienyl radical is optionally substituted with 1 to 3 substituent independently selected from a group consisting of fluoride, chloride, bromide, methyl, trifluoromethyl and methoxy;
R¹ represents hydrogen or a lower alkyl group from C₁ to C₄;
R² represents a dialkyl(C₁-C₄)aminoalkyl (C₂-C₃), or azaheterocyclylalkyl (C₂-C₃) radical; and HET represents a 5-membered heteroaryl radical, having from 1 to 3 nitrogen atoms as ring members and optionally substituted with 1 or 2 radicals independently selected from the group consisting of fluoride, chloride, bromide and methyl;
or a racemate, stereoisomer, enantiomer, or diastereomer, thereof, or a mixture of stereoisomers, enantiomers, or diastereomers thereof;
or an acid or base form, a salt, a solvate, or a hydrate thereof.

62. The method of claim 61, wherein R¹ is selected from the group consisting of hydrogen, methyl, ethyl, propyl, isopropyl, butyl, isobutyl, sec-butyl and tert-butyl.

63. The method of claim 61, wherein R² is selected from the group consisting of dimethylaminoethyl, dimethylaminopropyl, diethylaminoethyl, piperidinylethyl, morpholinylpropyl and pyrrolidinylethyl.

64. The method of claim 61, wherein said compound is of the formula (Ia) 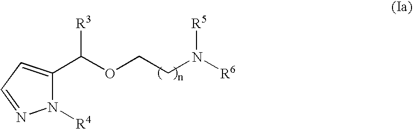
wherein
n is 1 or 2;
R³ is 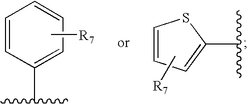
R⁴ is selected from the group consisting of hydrogen, fluoride, chloride, bromide and methyl;
R⁵ and R⁶ are independently lower C₍₁₋₄₎-alkyl or together with the nitrogen atom to which they are bonded form an azaheterocyclic ring;
R⁷ is selected from the group consisting of hydrogen, fluoride, chloride, bromide, methyl, trifluoromethyl and methoxy;
or an acid or base form, a salt, a solvate, or a hydrate thereof.

65. The method of claim 64, wherein R⁷ is hydrogen.

66. The method of claim 64, wherein R⁴ is methyl.

67. The method of claim 64, wherein R⁵ and R⁶ are CH₃ or C₂H₅ or together with the nitrogen atom to which they are bonded form a piperidinyl, morpholinyl or pyrrolidinyl ring.

68. The method of claim 64, wherein said compound is selected from the group consisting of:
5-{α-[2-(dimethylamino)ethoxy]benzyl}-1-methyl-1H-pirazole,
(±)-5-{α-[2-(dimethylamino)ethoxy]benzyl}-1-methyl-1H-pirazole,
(+)-5-{α-[2-(dimethylamino)ethoxy]benzyl}-1-methyl-1H-pirazole,
(−)-5-{α-[2-(dimethylamino)ethoxy]benzyl}-1-methyl-1H-pirazole,
5-{α-[2-(dimethylamino)ethoxy]-2-thienylmethyl}-1-methyl-1H-pirazole,
(±)-5-{α-[2-(dimethylamino)ethoxy]-2-thienylmethyl}-1-methyl-1H-pirazole,
(+)-5-{α-[2-(dimethylamino)ethoxy]-2-thienylmethyl}-1-methyl-1H-pirazole, and
(−)-5-{α-[2-(dimethylamino)ethoxy]-2-thienylmethyl}-1-methyl-1H-pirazole.

69. The method of claim 64, wherein said compound is of the formula (Ib) 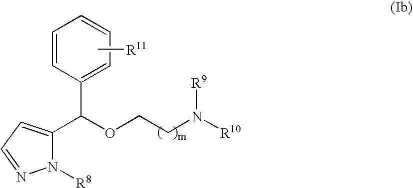
wherein
m is 1 or 2;
R⁸ is selected from the group consisting of hydrogen, fluoride, chloride, bromide and methyl;
R⁹ and R¹⁰ are independently lower C₍₁₋₄₎-alkyl or together with the nitrogen atom to which they are bonded form an azaheterocyclic ring;
R¹¹ is selected from the group consisting of hydrogen, fluoride, chloride, bromide, methyl, trifluoromethyl and methoxy;
or an acid or base form, a salt, a solvate, or a hydrate thereof.

70. The method of claim 69, wherein R¹¹ is hydrogen.

71. The method of claim 69, wherein R⁸ is methyl.

72. The method of claim 69, wherein R⁹ and R¹⁰ are CH₃ or C₂H₅ or together with the nitrogen atom to which they are bonded form a piperidinyl, morpholinyl or pyrrolidinyl ring.

73. The method of claim 69, wherein m is 1.

74. The method of claim 69, wherein said compound is selected from the group consisting of:
5-{α-[2-(dimethylamino)ethoxy]benzyl}-1-methyl-1H-pirazole,
(±)-5-{α-[2-(dimethylamino)ethoxy]benzyl}-1-methyl-1H-pirazole,
(+)-5-{α-[2-(dimethylamino)ethoxy]benzyl}-1-methyl-1H-pirazole,
(−)-5-{α-[2-(dimethylamino)ethoxy]benzyl}-1-methyl-1H-pirazole,
and citrate salts thereof.

75. The method of claim 64, wherein said compound is of the formula (Ic) 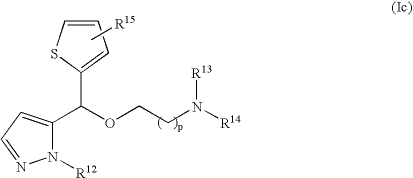
wherein
p is 1 or 2;
R¹² is selected from the group consisting of hydrogen, fluoride, chloride, bromide and methyl;
R¹³ and R¹⁴ are independently lower C₍₁₋₄₎-alkyl or together with the nitrogen atom to which they are bonded form an azaheterocyclic ring;
R¹⁵ is selected from the group consisting of hydrogen, fluoride, chloride, bromide, methyl, trifluoromethyl and methoxy.

76. The method of claim 75, wherein R¹⁵ is hydrogen.

77. The method of claim 75, wherein R¹² is methyl.

78. The method of claim 75, wherein R¹³ and R¹⁴ are CH₃ or C₂H₅ or together with the nitrogen atom to which they are bonded form a piperidinyl, morpholinyl or pyrrolidinyl ring.

79. The method of claim 75, wherein p is 1.

80. The method of claim 75, wherein said compound is selected from the group consisting of:
5-{α-[2-(dimethylamino)ethoxy]-2-thienylmethyl}-1-methyl-1H-pirazole,
(±)-5-{α-[2-(dimethylamino)ethoxy]-2-thienylmethyl}-1-methyl-1H-pirazole,
(+)-5-{α-[2-(dimethylamino)ethoxy]-2-thienylmethyl}-1-methyl-1H-pirazole,
(−)-5-{α-[2-(dimethylamino)ethoxy]-2-thienylmethyl}-1-methyl-1H-pirazole,
and citrate salts thereof.
